(19) United States
(12) Patent Application Publication
(10) Pub. No.: US 2026/0036529 A1
(43) Pub. Date: Feb. 5, 2026

(54) FLUID MONITORING AND CONTAMINANT DETECTION IN HYDRAULIC CYLINDERS USING HIGH-FREQUENCY ELECTROMAGNETIC SIGNALS

(71) Applicant: Pacoma GmbH, Eschwege (DE)

(72) Inventors: Guenter Leicht, Eschwege (DE); Karl-Wilhelm Herwig, Eschwege (DE); Martin Luessi, Portland, OR (US); Carsten Heckmann, Eschwege (DE)

(21) Appl. No.: 19/288,638

(22) Filed: Aug. 1, 2025

Related U.S. Application Data
(60) Provisional application No. 63/678,746, filed on Aug. 2, 2024.

(51) Int. Cl. G01N 22/04; G01N 22/02; G01N 33/28; G01S 13/88

(52) U.S. Cl. CPC G01N 22/04; G01N 22/02; G01N 33/2847; G01N 33/2888; G01S 13/88

(57) ABSTRACT

A method for monitoring a hydraulic fluid includes moving a piston to a defined position within a hydraulic cylinder and emitting, by a radar sensing unit, a radar signal through the hydraulic fluid in the hydraulic cylinder. The method also includes collecting, at the radar sensing unit, a reflected signal corresponding to the emitted radar signal. The method also includes comparing the reflected signal to a previously collected signal, wherein the previously collected signal was collected by the radar sensing unit while the piston was previously located at the defined position within the hydraulic cylinder. The method also includes identifying the presence of one or more contaminants in the hydraulic fluid based on the comparison.

### CROSS-REFERENCE TO RELATED APPLICATIONS

This application claims priority to U.S. Patent Application Ser. No. 63/678,746, filed Aug. 2, 2024, the entire contents of which are incorporated herein by reference.

### TECHNICAL FIELD

The description generally relates to hydraulic cylinders, for example, used in machinery and/or construction equipment.

### BACKGROUND

Hydraulic cylinders are hydraulic actuators that provide linear motion when hydraulic energy is converted into mechanical movement. Hydraulic cylinders get their power from pressurized hydraulic fluid. The hydraulic fluid is typically an incompressible (or a substantially incompressible) fluid. Over time, the hydraulic fluid may become contaminated due to various causes. Contamination of the hydraulic fluid inside a hydraulic cylinder can affect the performance of the hydraulic cylinder.

### SUMMARY

This document describes techniques for monitoring the condition of the fluid (e.g., a hydraulic fluid) in a hydraulic cylinder. Monitoring the condition of a hydraulic fluid can include detecting the presence or level of contaminants in the fluid, and can be performed using high-frequency electromagnetic signals (e.g., radar signals emitted by a radar sensing unit or microwave signals emitted by a microwave sensing unit). Contamination of the fluid can arise from various causes including the undesirable introduction of water or hard particles into the hydraulic cylinder, oxidation processes, etc. Since contamination can affect the time of flight of high-frequency electromagnetic signals within the hydraulic cylinder, measurements taken using a high-frequency electromagnetic sensing unit (e.g., a radar sensor operating in a microwave band, a millimeter wave band, etc.) can be used to monitor the fluid and detect contamination within the cylinder.

For simplicity, this specification describes example implementations of the invention primarily with reference to radar sensing units that emit and detect electromagnetic signals in the millimeter wave band (e.g., at approximately 60 GHZ). It should be understood, however, that the disclosed techniques are equally applicable to other suitable high-frequency electromagnetic signals including suitable signals in the microwave band.

In one aspect, a method for monitoring a hydraulic fluid is featured. The method includes moving a piston to a defined position within a hydraulic cylinder and emitting, by a radar sensing unit, a radar signal through the hydraulic fluid in the hydraulic cylinder. The method also includes collecting, at the radar sensing unit, a reflected signal corresponding to the emitted radar signal. The method also includes comparing the reflected signal to a previously collected signal, wherein the previously collected signal was collected by the radar sensing unit while the piston was previously located at the defined position within the hydraulic cylinder. The method also includes identifying the presence of one or more contaminants in the hydraulic fluid based on the comparing.

Implementations can include the examples described below and herein elsewhere. In some implementations, the one or more contaminants can include at least one of (i) water content in the hydraulic fluid, (ii) a presence of residues, or (iii) a presence of one or more particles in the hydraulic fluid. In some implementations, identifying the presence of one or more contaminants in the hydraulic fluid can include detecting a difference in a time-of-flight associated with the reflected signal and a time-of-flight associated with the previously collected signal, wherein the detected difference is greater than a threshold difference. In some implementations, identifying the presence of one or more contaminants in the hydraulic fluid can include detecting an attenuation in the reflected signal compared to the previously collected signal, wherein the attenuation is greater than a threshold attenuation level. In some implementations, moving the piston to the defined position within the hydraulic cylinder can include moving the piston to a position of maximum extension. In some implementations, identifying the presence of one or more contaminants in the hydraulic fluid can include accounting for one or more properties of the hydraulic fluid and/or a time when the hydraulic fluid was last replaced. In some implementations, the method can include replacing the hydraulic fluid within the hydraulic cylinder subsequent to identifying the presence of one or more contaminants in the hydraulic fluid.

In another aspect, a system is featured. The system includes a hydraulic cylinder including a piston, a hydraulic fluid within the hydraulic cylinder, a radar sensing unit, and a computing device. The computing device includes a memory configured to store instructions, and one or more processors configured to execute the instructions to perform operations. The operations include moving the piston to a defined position within the hydraulic cylinder and emitting, by the radar sensing unit, a radar signal through the hydraulic fluid in the hydraulic cylinder. The operations also include collecting, at the radar sensing unit, a reflected signal corresponding to the emitted radar signal. The operations also include comparing the reflected signal to a previously collected signal, wherein the previously collected signal was collected by the radar sensing unit while the piston was previously located at the defined position within the hydraulic cylinder. The operations also include identifying the presence of one or more contaminants in the hydraulic fluid based on the comparing.

Implementations can include the examples described below and herein elsewhere. In some implementations, the one or more contaminants can include at least one of (i) water content in the hydraulic fluid, (ii) a presence of residues, or (iii) a presence of one or more particles in the hydraulic fluid. In some implementations, identifying the presence of one or more contaminants in the hydraulic fluid can include detecting a difference in a time-of-flight associated with the reflected signal and a time-of-flight associated with the previously collected signal, wherein the detected difference is greater than a threshold difference. In some implementations, identifying the presence of one or more contaminants in the hydraulic fluid can include detecting an attenuation in the reflected signal compared to the previously collected signal, wherein the attenuation is greater than a threshold attenuation level. In some implementations, moving the piston to the defined position within the hydraulic cylinder can include moving the piston to a position of maximum extension. In some implementations, identifying the presence of one or more contaminants in the hydraulic fluid can include accounting for one or more properties of the hydraulic fluid and/or a time when the hydraulic fluid was last replaced. In some implementations, the operations can include replacing the hydraulic fluid within the hydraulic cylinder subsequent to identifying the presence of one or more contaminants in the hydraulic fluid.

In another aspect, one or more machine-readable storage devices are featured. The one or more machine-readable storage devices have encoded thereon computer readable instructions for causing one or more processing devices to perform operations. The operations include moving the piston to a defined position within the hydraulic cylinder and emitting, by the radar sensing unit, a radar signal through the hydraulic fluid in the hydraulic cylinder. The operations also include collecting, at the radar sensing unit, a reflected signal corresponding to the emitted radar signal. The operations also include comparing the reflected signal to a previously collected signal, wherein the previously collected signal was collected by the radar sensing unit while the piston was previously located at the defined position within the hydraulic cylinder. The operations also include identifying the presence of one or more contaminants in the hydraulic fluid based on the comparing.

Implementations can include the examples described below and herein elsewhere. In some implementations, the one or more contaminants can include at least one of (i) water content in the hydraulic fluid, (ii) a presence of residues, or (iii) a presence of one or more particles in the hydraulic fluid. In some implementations, identifying the presence of one or more contaminants in the hydraulic fluid can include detecting a difference in a time-of-flight associated with the reflected signal and a time-of-flight associated with the previously collected signal, wherein the detected difference is greater than a threshold difference. In some implementations, identifying the presence of one or more contaminants in the hydraulic fluid can include detecting an attenuation in the reflected signal compared to the previously collected signal, wherein the attenuation is greater than a threshold attenuation level. In some implementations, moving the piston to the defined position within the hydraulic cylinder can include moving the piston to a position of maximum extension. In some implementations, identifying the presence of one or more contaminants in the hydraulic fluid can include accounting for one or more properties of the hydraulic fluid and/or a time when the hydraulic fluid was last replaced. In some implementations, the operations can include replacing the hydraulic fluid within the hydraulic cylinder subsequent to identifying the presence of one or more contaminants in the hydraulic fluid.

Various implementations of the technology described herein may provide one or more of the following advantages. Radar sensors used in or with hydraulic cylinders can serve multiple purposes including measuring the location of a piston within the hydraulic cylinder. Thus, the techniques described herein can enable fluid monitoring and contaminant detection while requiring little to no additional hardware compared to other systems that use radar sensors for collecting piston position measurements. In addition, by detecting contamination in the hydraulic fluid, the techniques described herein can have the advantage of notifying an owner or operator of the hydraulic cylinder to replace the hydraulic fluid and/or the hydraulic cylinder when necessary, thereby enabling improved performance and improved maintenance of hydraulic cylinders (and related systems). The techniques described herein can further provide the advantage of being usable in high-pressure settings, which can allow for fluid monitoring in the hydraulic cylinder itself. In contrast, conventional techniques for fluid monitoring are typically suited for low pressure settings only, and are therefore limited to detecting contaminants in a tank that supplies the hydraulic fluid to the hydraulic cylinder (rather than detecting contaminants directly within the hydraulic cylinder).

Other features and advantages of the description will become apparent from the following description, and from the claims. Unless otherwise defined, the technical and scientific terms used herein have the same meaning as commonly understood by one of ordinary skill in the art to which this invention belongs.

### BRIEF DESCRIPTION OF DRAWINGS

FIG. 1A shows a piston and cylinder unit with an uncontaminated hydraulic fluid, and associated measurements from a radar sensing unit.

FIG. 1B shows a piston and cylinder unit with a contaminated hydraulic fluid, and associated measurements from a radar sensing unit.

FIG. 2 shows a longitudinal section of a first example of a piston and cylinder unit.

FIG. 3 shows a partial longitudinal section of a second example of a piston and cylinder unit in the area of the cylinder head.

FIG. 4 shows an exploded view of the cylinder head shown in FIG. 3.

FIG. 5 shows a three-dimensional view of a piston movement sensor and a

positioning and/or alignment element.

FIG. 6 shows a spatial view of a housing connector with a sensor cable.

FIG. 7 shows the housing connector with sensor cable according to FIG. 6 in a side view.

FIG. 8 shows a three-dimensional view of a housing plug with a sensor cable.

FIG. 9 shows the housing connector according to FIG. 8 in a side view.

FIG. 10A shows a longitudinal section of a third example of a piston and cylinder unit.

FIG. 10B is a close-up view of the longitudinal section of the piston and cylinder unit shown in FIG. 10A.

FIGS. 11A-11C are plots showing a relationship between measurements taken by a radar sensing unit in a cylinder and levels of water contamination in a mineral oil-based hydraulic fluid (HLP46) within the cylinder.

FIG. 12A-12C are plots showing a relationship between measurements taken by a radar sensing unit in a cylinder and levels of water contamination in a biodegradable PAO-based hydraulic fluid (AVIA Syntofluid PE-B 50) within the cylinder.

FIG. 13 is a flowchart showing a process for monitoring a hydraulic fluid.

FIG. 14 is a diagram illustrating an example of a computing environment.

FIG. 15 is a cross-sectional view of an example sensor assembly block.

FIG. 16 is an exploded view of the sensor assembly block of FIG. 15.

FIG. 17 is a close up view of a sensor housing unit, spacer, and a dielectric lens for the sensor assembly block of FIGS. 15 and 16.

FIG. 18A shows a close-up view of the spacer and a top view of the sensor housing unit, of FIGS. 15, 16, and 17.

FIG. 18B shows a cross-sectional view of the spacer and the sensor housing unit of FIG. 10A.

FIG. 19 illustrates an example system 1900 for identifying features of a fluid 1904 in a container 1902.

### DETAILED DESCRIPTION

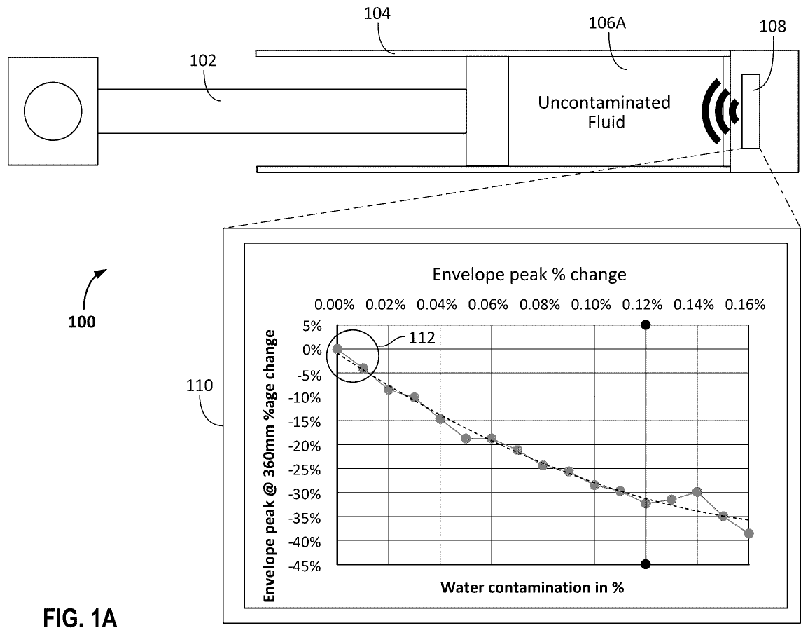

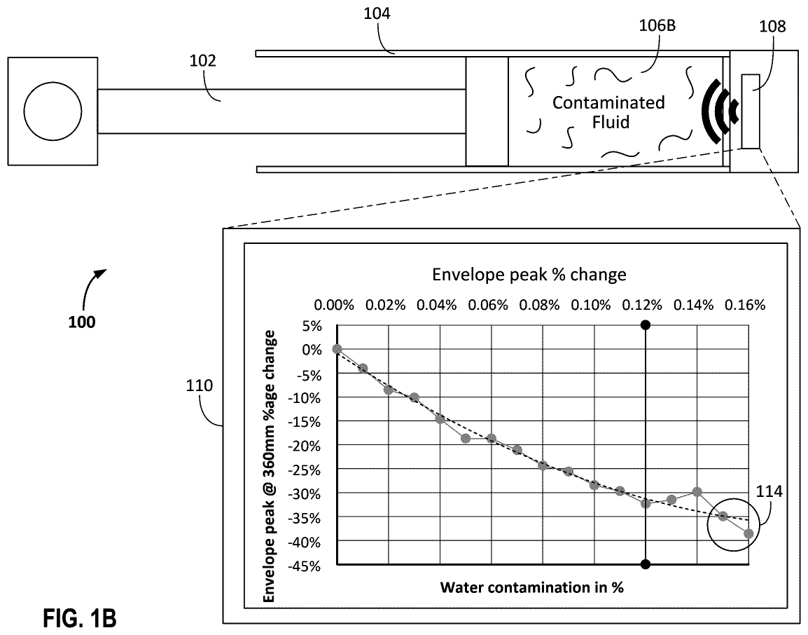

FIGS. 1A and 1B each show a piston and cylinder unit 100 with associated measurements from a radar sensing unit. The piston and cylinder unit 100, for example, can make up a hydraulic cylinder system, such as those used in industrial equipment including construction vehicles, manufacturing machinery, elevators, etc. The piston and cylinder unit 100 includes a piston 102 and a cylinder 104. Inside the cylinder 104, there is hydraulic fluid, which is uncontaminated in FIG. 1A (e.g., uncontaminated fluid 106A) and contaminated in FIG. 1B (e.g., contaminated fluid 106B). For example, the hydraulic fluid can be an oil (e.g., a mineral oil-based hydraulic fluid) or a biodegradable hydraulic fluid (e.g., a polyalphaolefin [PAO]-based fluid). As described in further detail herein, contamination of the hydraulic fluid can come in various forms including the introduction of water or hard particles into the piston and cylinder unit 100, residues resulting from chemical processes such as oxidation or corrosion, etc.

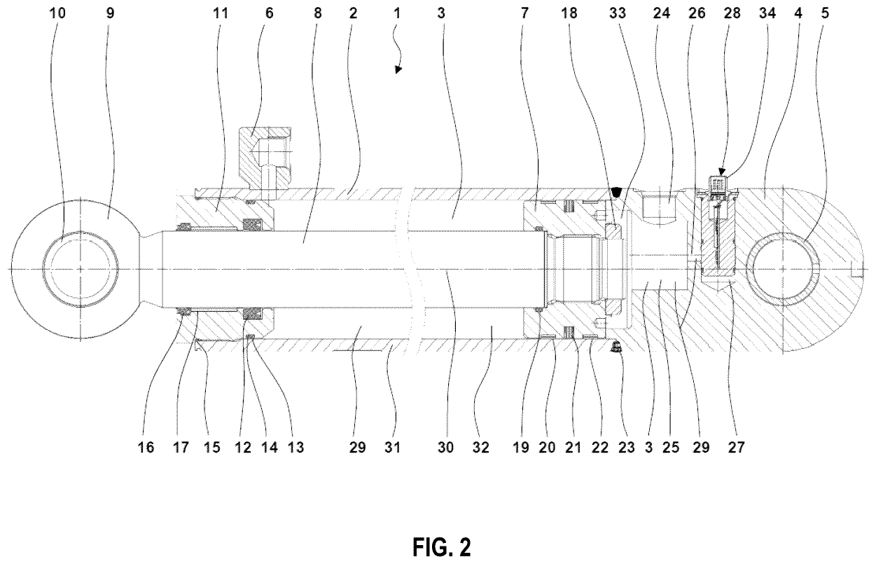

The piston and cylinder unit 100 further includes a radar sensing unit 108. The radar sensing unit 108 can include one or more radar emitters and/or one or more radar sensors. The one or more radar emitters can be configured to emit radar signals into the cylinder 104, e.g., through the hydraulic fluid within the cylinder 104. The radar signals travel through the hydraulic fluid, and reflect off the cylinder walls as well as from the head of the piston 102. The one or more radar sensors of the radar sensing unit 108 can then detect the reflected radar signals and convert the detected signal to an electrical signal. In some cases, the radar sensing unit 108 can include or be connected to one or more computing devices located within the cylinder 104 (or disposed remotely from the piston and cylinder unit 100 in distributed processing implementations) to analyze the resulting electrical signal to determine a position of the piston 102. In this way, the piston and cylinder unit 100 is able to use radar sensing techniques to measure and track the position of the piston 102 within the cylinder 104. Example embodiments of the piston and cylinder units with radar sensing units are described in further detail herein, for example, in relation to FIGS. 2-9 and FIGS. 10A-10B.

Over time and with operation of the piston and cylinder unit 100, the uncontaminated fluid 106A shown in FIG. 1A can become contaminated, resulting in the contaminated fluid 106B shown in FIG. 1B. This, in turn, can affect the measurements of the position of the piston 102 obtained by the radar sensing unit 108. For example, the plot 110 shows various position measurements obtained by the radar sensing unit 108 for the piston 102 at the same location, but with varying levels of water contamination present in the hydraulic fluid. At low levels of contamination, the hydraulic fluid represents the uncontaminated fluid 106A shown in FIG. 1A and yields relatively high peak signal measurements 112 corresponding to the particular piston location. On the other hand, at high levels of contamination, the hydraulic fluid represents the contaminated fluid 106B shown in FIG. 1B and yields relatively low peak signal measurements 114 corresponding to the particular piston location. This change in signal measurements (despite the piston being located in the same position) can be indicative of the presence of contaminants in the hydraulic fluid and can therefore be used to detect contamination and to identify when the hydraulic fluid (or the entire piston and cylinder unit 100) should be replaced. For example, in some implementations, a water content of about 0.12% (e.g., 0.08%, 0.10%, 0.14%, 0.16% by volume, etc.) can be considered a maximum acceptable limit of contamination within the hydraulic fluid, and it can be desirable to replace the hydraulic fluid before this critical level of water content is reached. In this way, improved performance and improved maintenance of piston and cylinder units can be achieved through effective contamination detection.

The change in signal measurements caused by contamination of the hydraulic fluid in the piston and cylinder unit 100 can be characterized based on physical properties of the piston and cylinder unit 100 as well as the radar signals that travel through the hydraulic fluid. An electromagnetic wave's velocity, travelling through a medium, depends on the medium's relative permittivity (εᵣ) or “dielectric constant.” Consequently, changes in the medium's εᵣ due to contamination can influence the radar sensing unit 108's ranging measurement. Contamination in hydraulic systems such as water, residues, and hard particles introduced by wear from seals, metals, etc. can all affect the εᵣ of the hydraulic fluid in the piston and cylinder unit 100, thereby affecting measurements obtained by the radar sensing unit 108. Residues can form in the hydraulic fluid due to chemical processes, e.g., oxidation, corrosion, environmental factors, improper maintenance, oil degradation (e.g., occuring from prolonged operation of the hydraulic cylinder) among other sources of residue in hydraulic fluids.

One type of contamination in hydraulic systems is water, which can be highly undesirable if present in the hydraulic fluid. Water can cause emulsions to form and can lead to corrosion of the piston and cylinder unit 100. Because water has a very high εᵣ of about 80 (compared to about 2.2 for mineral oil-based and PAO-based hydraulic fluids), even very small amounts of water content in the hydraulic fluid can increase the εᵣ of the fluid medium and therefore affect the ranging measurements obtained by the radar sensing unit 108. In addition, water is a polar fluid due to the dipole-characteristic of its molecules. Thus, in addition to causing a change in εᵣ, the presence of water in the cylinder 104 can result in substantial absorption and attenuation of millimeter wave or microwave energy while electromagnetic waves travel through a hydraulic fluid contaminated with water.

Another type of contamination in hydraulic systems is oxidation. Signs of the natural process of oxidation in the piston and cylinder unit 100 include changes in fluid color, odor, and/or acidity level of the hydraulic fluid medium. Sludge, gum, or varnish in the piston and cylinder unit 100 are further evidence that oxidation has taken place and may change the contaminated hydraulic fluid's εᵣ as well as the amount of energy absorption (e.g., attenuation) for the radar signal travelling through the fluid.

Yet another type of contamination in hydraulic systems is the presence of hard particles. Hydraulic pumps and servo valves in a hydraulic system can be damaged by fluid contaminated with hard particles larger than the clearance between lubricated surfaces. In addition, the presence of hard particles in the hydraulic fluid may change the εᵣ of the hydraulic fluid and/or the transparency of the hydraulic fluid to a radar signal (e.g., resulting in greater attenuation of the radar signal as it travels through the fluid).

The contamination types mentioned above (e.g., water, oxidation, and hard particles) all contribute to changes in the hydraulic fluid's εᵣ and/or attenuation of the electromagnetic wave travelling through the hydraulic fluid, and as a result, they all influence the ranging measurements obtained by the radar sensing unit 108. Thus, these changes in ranging measurements can be used for online contamination detection of the hydraulic fluid in the hydraulic cylinder, as described in further detail herein. By performing contamination detection using hardware already included in piston and cylinder units (e.g., radar sensing units used for piston position detection), the radar-based techniques for contamination detection disclosed herein can mitigate the need for a separate dedicated fluid condition sensor. Furthermore, the radar-based techniques disclosed herein can be advantageous since they enable the detection of contamination directly within the cylinder 104 itself (e.g., as opposed to within a tank that supplies hydraulic fluid to the cylinder 104) without directly subjecting the hardware of the radar sensing unit 108 to the high pressures within the cylinder 104.

In embodiments of the piston and cylinder units 100 described in this specification, the radar measurement of the piston position is performed by measuring the time it takes for the radar signal to travel from the sensor (e.g., part of the radar sensing unit 108) to the piston 102 and back. This time-of-flight measurement is dependent on the position of the piston 102 and the propagation velocity of the radar signal in the hydraulic fluid.

The propagation velocity (v) of the radar signal in a medium is described by v=c/n where c is the speed of light in vacuum and n is the refractive index. The refractive index (n) can be found using the equation n=\sqrt{εᵣμᵣ}, where μᵣ is the dielectric constant and ur is the magnetic constant of the material (in this case, the hydraulic fluid). For most non-magnetic materials, μᵣ≅1 and therefore n≅εᵣ. Therefore, by measuring the propagation velocity (v) of the radar signal, the dielectric constant of the material can be calculated using the equation, εᵣ=(c/v)². This calculation of the dielectric constant based on the propagation velocity of the radar signal (e.g., calculated using time-of-flight measurements) can be used to estimate a level of fluid contamination within a cylinder unit. For example, changes to the calculated dielectric constant over time can be indicative of fluid contamination. In some cases, rather than calculating the dielectric constant, the time-of-flight measurements and/or propagation velocity of the radar signal can be used as proxies for the dielectric constant and can be directly monitored to detect fluid contamination. In some embodiments, the monitoring of time-of-flight measurements, the propagation velocity of the radar signal, and/or the dielectric constant of the medium through which the radar signal travels can be implemented in addition to or in lieu of the attenuation-based methods of detecting fluid contamination described elsewhere in this specification.

As the time-of-flight of the radar signal is dependent on both the position of the piston and the propagation velocity, the position of the piston must be known in order to estimate the propagation velocity. This can be achieved by moving the piston to a known position when performing the measurement. A typical measurement position is the maximum position (e.g., the position that maximizes the distance between the piston 102 and the radar sensing unit 108 shown in FIGS. 1A and 1B), but other positions could be used as well, as long as the actual position of the piston is known.

Other factors, such as the temperature of the fluid and the pressure can also affect the dielectric constant and should preferably be taken into account when determining the condition of the fluid based on the dielectric constant. Therefore, in some implementations, temperature and/or pressure calibration techniques can be implemented to remove any changes in the radar signal measurements caused by temperature and/or pressure variations in the cylinder 104. In some implementations, to enable temperature calibration, the temperature within the cylinder 104 can be determined using a dedicated temperature sensor that can be, for example, included in the radar sensing unit 108. Even if the temperature sensor is not disposed directly within the hydraulic fluid, a temperature sensor included with the radar sensing unit 108 can still be effective in approximating the temperature within the cylinder for the purposes of acting as a feedback signal and/or input to a temperature calibration process.

In addition to the dielectric constant, the radar sensing unit 108 can also measure the rate of absorption of the radar signal by measuring the power of the returned signal. When obtained at a known piston position, this measure can be used as an additional parameter for determining the fluid condition.

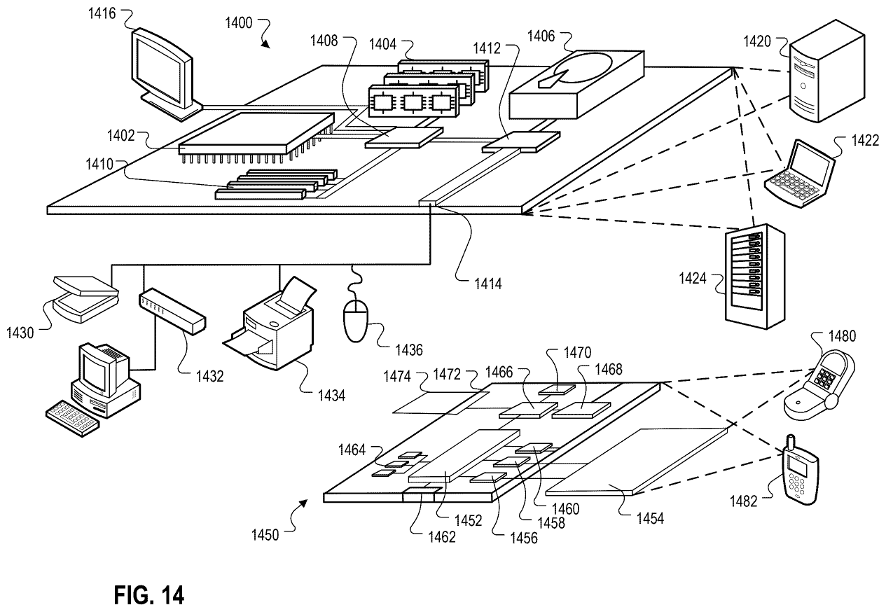

By periodically measuring the dielectric constant and/or the rate of absorption using the radar sensing unit 108 and then storing this information in memory (either internal or external to the piston and cylinder unit 100), a historical record of measurements can be compiled. These historical measurements can be analyzed (e.g., using computer software) to determine how the measurements changed over time to determine the fluid condition. In some implementations, additional information such as the type of hydraulic fluid and when the fluid was last changed can also be considered (e.g., by the computer software) to further improve the estimation of the fluid quality. In general, any software used to perform this analysis can be performed using one or more processors disposed at the piston and cylinder unit 100 itself (e.g., as part of the radar sensing unit 108), at one or more external computing devices that communicate with the radar sensing unit 108 (e.g., over a data bus such as a CAN bus), or both. Examples of computing devices that can implement such software are described in further detail in relation to FIG. 14 below.

In some implementations, it is possible not only to detect the presence of contamination, but also to identify a particular instance and type of contaminant based on time-of-flight measurements, the measured dielectric constant, the rate of absorption, and/or other characteristics of the radar signal measurements obtained by the radar sensing unit 108. For example, by training a machine learning model on historical measurements associated with different kinds of contamination, one or more trained machine learning models can be developed to analyze the measured dielectric constant, the rate of absorption, and/or other characteristics of the radar signal measurements obtained by the radar sensing unit 108 to classify an instance of detected contamination as a particular type of contamination (e.g., water versus oxidation versus hard particles). In some implementations, other characteristics of the contamination instance can be determined including, for example, a level of dispersion of contaminants within the hydraulic fluid.

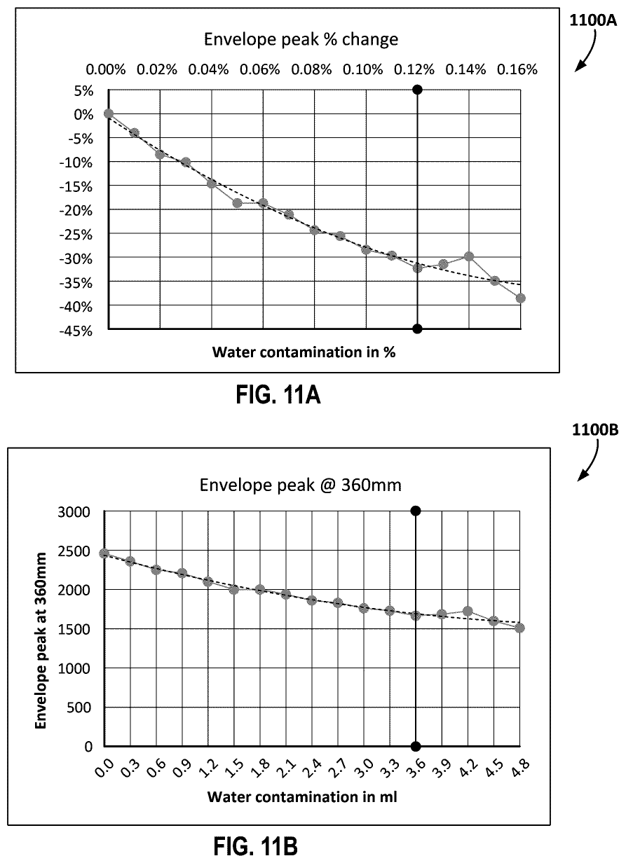

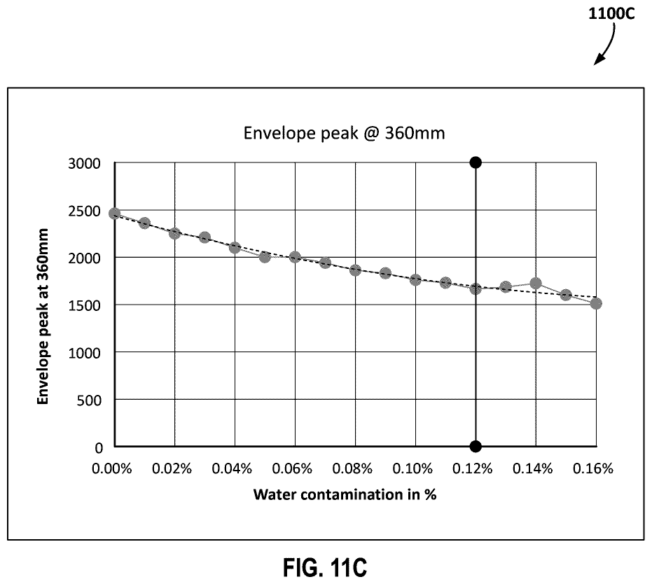

Examples of pistons and cylinder units are described in U.S. Pat. No. 11,378,107, U.S. PG Publication No. 2022/0057477 A1, and EP 4 246 001 A1 which are incorporated herein by reference in their entirety. Such pistons and cylinder units can be used to implement the contamination detection techniques described herein. For example, FIGS. 2-9 illustrate different views of illustrative embodiments of a piston and cylinder unit 1. FIGS. 10A-10B illustrate different views of yet another embodiment of a piston and cylinder unit 1001.

An example piston and cylinder unit is described in the publication DE 10 2019 122 121 A1. This piston and cylinder unit 1 (sometimes referred to herein as “piston-cylinder unit” 1) is shown in FIG. 2. In FIG. 2, it is shown by means of break lines that the piston-cylinder unit I can actually be longer and that only part of the piston-cylinder unit 1 is shown. The piston-cylinder unit 1 has a cylinder 2 with a cylinder tube 31, an interior 3 and a cylinder head 4. The cylinder tube 31 is connected to the cylinder head 4 via a weld scam 23. A bearing bush 5 is arranged in the area of the cylinder head 4. In DE 10 2019 122 121 A1, the piston-cylinder unit 1 is a hydraulic piston-cylinder unit, so the interior 3 is filled with a hydraulic fluid 29, in particular oil. For this purpose, the cylinder 2 has a connection 6 and a connection 24. A hydraulic circuit, not shown here, with a hydraulic pump and changeover valves is connected to the connections 6, 24. The connections 6, 24 each open into an associated pressure chamber 32, 33. The pressure chambers 32, 33 are formed in the interior 3 and separated from one another by a piston 7. The piston 7 can be moved along the longitudinal central axis 30 of the cylinder 2 while sealing the pressure chambers 32, 33. Depending on the pressure generated by the hydraulic circuit at the connections 6, 24, an actuating force can be generated hydraulically in both directions along the longitudinal central axis 30, which acts on the piston 7, and thereby generates movement of the piston 7 and a change in the volume of the pressure chambers 32, 33.

FIG. 2 shows the position of the piston 7 moved all the way to the right, in a fully retracted position of the piston-cylinder unit 1. The piston 7 is connected to a piston rod 8, at the outer end of which a piston rod eye 9 is arranged. The piston rod eye 9 also has a bearing bush 10. The bearing bushes 5, 10 serve to link the piston-cylinder unit 1 to parts of a work machine that are to be moved relative to one another by means of the piston-cylinder unit 1 and/or on which the piston-cylinder unit 1 exerts a force. The piston rod 8 is mounted in a translationally movable manner in the axial direction along the longitudinal central axis 30 by means of a guide bushing 11. A rod seal 12, an O-ring 13 and a support ring 14 are provided for support and sealing. At the other axial end of the guide bushing 11, another O-ring 15, a wiper 16 and a plain bearing 17 are arranged. The piston 7 is arranged non-rotatably on the piston rod 8 and secured by means of a lock nut 18. Furthermore, an O-ring 19, a piston guide ring 20, a piston seal 21 and a further piston guide ring 22 are arranged on the piston 7. In this way, the piston 7 is mounted together with the piston rod 8 and the piston rod eye 9 in a translationally reciprocating and sealing manner in the cylinder tube 31 of the cylinder 2. A partial chamber 25 of the pressure chamber 33 in the cylinder head 4 adjoins the part of the pressure chamber 33 which is delimited by the cylinder tube 31. The partial chamber 25 is connected to the connection 24. An axially extending sensor signal channel 26 opens into this partial chamber 25. The sensor signal channel 26 is also part of the pressure chamber 33 and is therefore filled with the hydraulic fluid. The sensor signal channel 26 is in turn connected to a transverse bore 27 which extends radially to the longitudinal central axis 30 in the cylinder head 4. The transverse bore 27 extends to the outer surface of the cylinder head 4 and can be connected to the environment by means of a compensating bore (not shown).

A piston movement sensor 28 (also referred to as a piston position detection unit 28) is arranged in the transverse bore 27. The piston movement sensor 28 is used to detect the axial position of the piston 7 in the cylinder 2 using high-frequency technology (e.g., using radar signals). For example, the piston movement sensor 28 can be a radar sensing unit (such as radar sensing unit 108) including one or more radar sensors and/or emitters configured to emit radar signals into the cylinder 2 and detect reflected radar signals. For this purpose, the piston movement sensor 28 emits a high-frequency signal, which hits the end face of the piston 7 or the piston rod 8 through the sensor signal channel 26 and through the partial chamber 25 as well as through the pressure chamber 33 and, after reflection through this end face, returns to the piston movement sensor 28. The movement signal, in particular the path traveled by the end face, can then be determined from the reflected signal using high-frequency technology, in particular by evaluating the transit time (time of flight). For example, an electronic unit connected to or included in the piston movement sensor 28 (including electronic components and software executed by these components) can carry out an evaluation of the reflected signals to determine the current position of the piston 7 along the longitudinal central axis 30. This determination can be conducted permanently, in defined time intervals or at specific points in time. In some implementations, the result or a command being associated with the result is transmitted to an electronic computing unit of the working machine connected therewith—a part of which is the piston and cylinder unit 1.

In the embodiment shown in FIG. 2, the piston movement sensor 28 is acted upon by the hydraulic fluid. A sensor housing of the piston movement sensor 28 has seals with which the piston movement sensor 28 is sealed axially on both sides of the sensor signal channel 26 so that the hydraulic fluid cannot escape from the pressure chamber 33 and via the transverse bore 27. The piston movement sensor 28 here has a connection plug 34, which is carried by the sensor housing of the piston movement sensor 28 and extends radially out of the cylinder head 4. For further details, reference is made to the publication DE 10 2019 122 121 A1, which is incorporated herein by reference in its entirety.

A further development of the piston-cylinder unit 1 is known from the publication EP 3 957 868 A1. It is proposed here that a collimator is arranged in the beam path for the high-frequency signal, which serves to increase the measurement accuracy of the piston movement sensor. A collimator is understood to be an optical device for generating a beam path with parallel beams from previously non-parallel beams from divergent sources. In a first direction of radiation from a transmitting unit of the piston movement sensor to the end face of the piston or the piston rod, the collimator converts the non-parallel rays emitted by the piston movement sensor into parallel rays, which are then also reflected in parallel from the end face of the piston or the piston rod. The reflected high-frequency beams are then bundled again by the collimator in the opposite second direction of radiation so that they can be received and evaluated by a receiving unit of the piston movement sensor. The collimator can also act as a type of filter that only or essentially focuses the high-frequency beams onto the piston movement sensor, which previously ran parallel to each other and to the longitudinal axis of the piston. This makes it possible to filter out high-frequency rays that do not come from the end piston crown surface, or at least not directly. Such undesirable rays are due to the fact that in reality the refraction of the collimator is not ideal, the rays are not emitted and received in an ideal point manner and the piston bottom surface is not ideally flat. The use of the collimator is intended to improve the signal-to-noise ratio. The collimator may include a dielectric lens. It is also possible to use several dielectric lenses or a Fresnel zone plate. The dielectric lens can have a convexly curved lens surface and/or be made of material from a dielectric plastic or a dielectric ceramic, polytetrafluoroethylene, polyethylene or polypropylene. The dielectric lens preferably has a dielectric constant (permittivity) greater than that of air and greater than that of the hydraulic fluid in the piston-cylinder assembly. The permittivity can be, for example, between 20% and 50% greater than that of the hydraulic fluid in the piston-cylinder unit. The permittivity difference and the curvature of the dielectric lens are coordinated with one another. The dielectric lens may have a planar-convex lens shape. The convex side of the lens can face the piston. On the other hand, the planar side then faces the piston movement sensor. The collimator may be formed by the sensor housing or may be structurally separated from the piston movement sensor itself and the sensor housing. The piston movement sensor can also be designed as a compact built-in cartridge that contains both the sensor and the evaluation electronics. The piston movement sensor is arranged in the cross bore with an orientation such that the longest dimension of the piston movement sensor extends in the direction of the longitudinal axis of the cross bore. Away from the sensor signal channel, beam deflection elements can be arranged on a bottom of the partial chamber in order to avoid falsification of the measurement results. The collimator can be arranged in the sensor signal channel. For further details, reference is made to the publication EP 3 957 868 A1, which is herein incorporated by reference in its entirety.

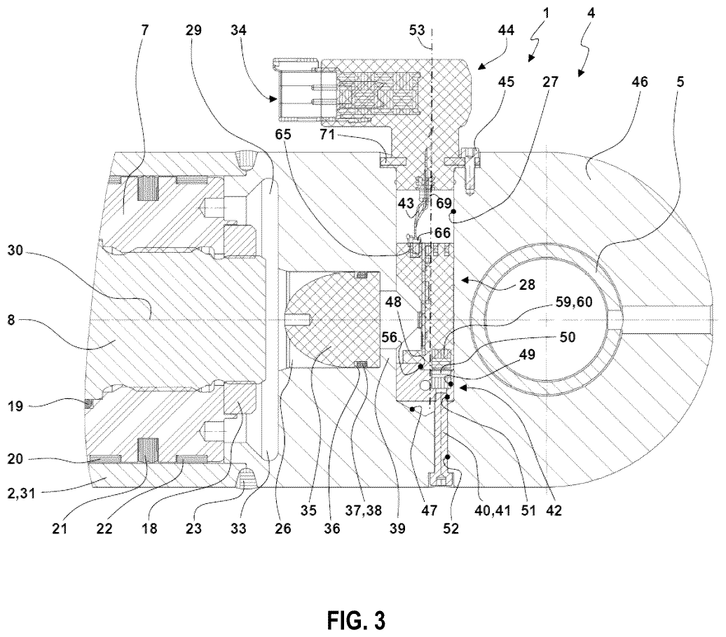

FIGS. 3-9 show another embodiment of a piston and cylinder unit 1, as described and shown in publication EP 4 246 001 A1, which is herein incorporated by reference in its entirety. The embodiment shown in FIGS. 3-9 has many similarities to the embodiment shown in FIG. 2, with similar elements labeled using similar reference numerals. Except where otherwise stated, what has been described about the embodiment shown in FIG. 2 is also applicable to the embodiment shown in FIGS. 3-9, and the further disclosure in the publications DE 10 2019 122 121 A1 and EP 3 957 868 A1 can also be used within the scope of these embodiments.

FIG. 3 shows a piston-cylinder unit 1 in the area of the cylinder head 4. A sensor signal channel 26 opens into the pressure chamber 33 of the piston-cylinder unit 1. A collimator 35 is arranged in the sensor signal channel 26. The collimator 35 has a flat end face on the side facing the piston movement sensor 28, which is oriented transversely to the longitudinal central axis 30. With regard to the longitudinal central axis 30, the collimator 35 is designed to be rotationally symmetrical on the other side. The collimator 35 can, for example, have a curved and in particular parabolic longitudinal section, as shown. The collimator 35 has an annular groove 36 in which a sealing element 37, here an O-ring 38, is arranged. The sealing element 37 ensures a hydraulic seal between the inner wall of the sensor signal channel 26 and the collimator 35. The sensor signal channel 26 has a circumferential shoulder 39. If the pressure chamber 33 is pressurized with hydraulic pressure, the pressure acts on the spherical end face facing the piston 7, applying a hydraulic force that presses the collimator 35 against the shoulder 39. This pressure of the collimator 35 on the shoulder 39 and/or the effect of the sealing element 37 can ensure that the transverse bore 27 is not exposed to hydraulic fluid and therefore no additional sealing measures need to be taken in the transverse bore 27. On the other hand, this seal makes it possible to dismantle the piston movement sensor 28 without hydraulic fluid being able to escape from the transverse bore 27.

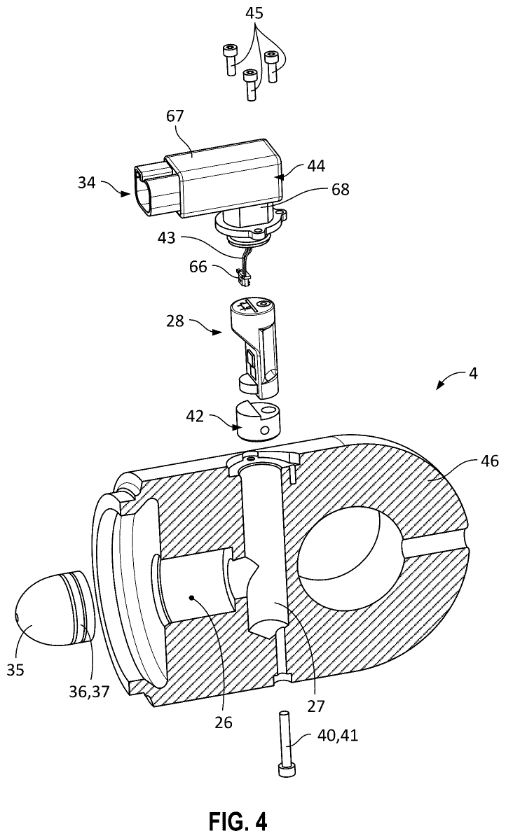

As shown in the exploded view in FIG. 4, a securing clement 40 in the form of a screw 41, a positioning and/or alignment element 42, the piston movement sensor 28, a sensor cable 43 and a housing plug 44 are mounted in the transverse bore 27, the housing plug 44 being attached to the housing 46 of the cylinder head 4 via fastening screws 45.

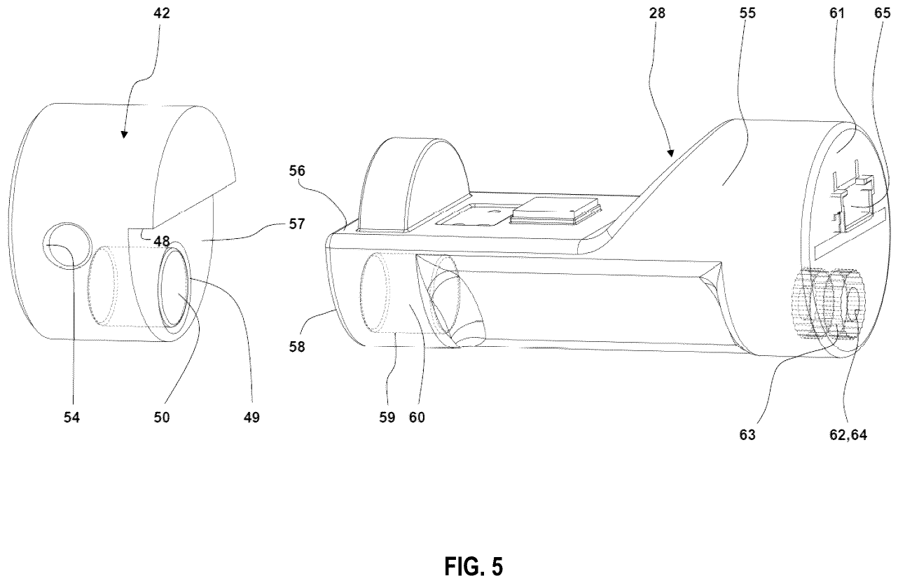

According to FIG. 5, the positioning and/or alignment element 42 is cylindrical with a diameter such that the positioning and/or alignment element 42 can be inserted precisely into the transverse bore 27. The underside of the positioning and/or alignment clement 42 is flat for the exemplary embodiment shown. The underside of the positioning and/or alignment element 42 rests on a bottom 47 of the transverse bore 27, which is designed here as a blind hole.

On the side facing the piston movement sensor 28, the positioning and/or alignment element 42 is basically flat, but is designed with a step 48. On this side, the positioning and/or alignment element 42 has a (here cylindrical) receptacle 49, in which a permanent magnet 50 is accommodated, which can be glued to the receptacle 49 or pressed into it. The outer surface of the permanent magnet 50 is arranged flush with a partial surface of the end face of the positioning and/or alignment clement 42 away from the step 48.

On the side facing away from the piston movement sensor 28, the positioning and/or alignment element 42 has an internal thread 51 arranged eccentrically to the longitudinal axis 53 of the transverse bore 27. In the aligned position of the positioning and/or alignment element 42 installed in the transverse bore 27, is an aligned internal thread 51 of the positioning and/or alignment element 42 with an eccentric bore 52 opening into the transverse bore 27. It is through this opening that the screw 41 extends from the outside through the housing 46 to fix the positioning and/or alignment element 42 in the correct position and orientation.

It is possible that the positioning and/or alignment element 42 also has a transverse bore 54, possibly with an internal thread. The transverse bore 54 is unlike the bore 52 shown in FIG. 3, which is oriented parallel to the longitudinal axis 53 of the transverse bore 27. Rather the transverse bore 54 is a hole provided perpendicularly to the plane of the drawing in which FIG. 3 is oriented. As an alternative to the attachment via the screw 41, the positioning and/or alignment element 42 can be attached via a screw which is perpendicular to the plane of the drawing in which FIG. 3 is oriented. The screw can extend through the housing 46 and is screwed in the inner end region to the transverse bore 54 of the positioning and/or alignment element 42.

The piston movement sensor 28 has a sensor housing 55, the external geometry of which is cylindrical with a diameter such that the sensor housing 55 can find a precise fit in the transverse bore 27. The sensor housing 55 also has recesses in which electronic components and the transmitting and/or receiving unit for the high-frequency signal are arranged.

On the side facing the positioning and/or alignment element 42, the sensor housing 55 has a step 56 which is designed to correspond to the step 48 of the positioning and/or alignment element 42. Away from the steps 48, 56, the positioning and/or alignment element 42 and the sensor housing 55 form contact surfaces 57, 58 which are oriented transversely to the longitudinal axis 53. The area in which these contact surfaces 57, 58 rest against one another in the direction of the longitudinal axis 53 defines the axial position of the piston movement sensor 28.

The steps 48, 56 further form a fit that prevents rotation about the longitudinal axis 53 and determines the orientation of the piston movement sensor 28. In the relative orientation determined by the steps 48, 56, a corresponding receptacle 59 with a permanent magnet 60 is provided on the sensor housing 55, aligned with the receptacle 49 and the permanent magnet 50 of the positioning and/or alignment element 42. The permanent magnet 60 is also fixed in the receptacle 59, for example by gluing or pressing it in. The magnetic force between the permanent magnets 50, 60 secures the system and thus the position and alignment between the positioning and/or alignment element 42 and the piston movement sensor 28.

On the side facing away from the positioning and/or alignment element 42, the sensor housing 55 has a flat end face 61. In the vicinity of the end face 61, the piston movement sensor 28 has an internal thread 62, which is formed here by a thread insert 63 injected into the sensor housing 55. The internal thread 62 forms a dismantling driver 64.

Furthermore, a plug receptacle 65 is provided in the end face 61, into which a plug 66 of a sensor cable 43 can be inserted. In some implementations, the format of the plug 66 and the plug receptacle 65 is a 5-pin pico-clasp connection (registered trademark).

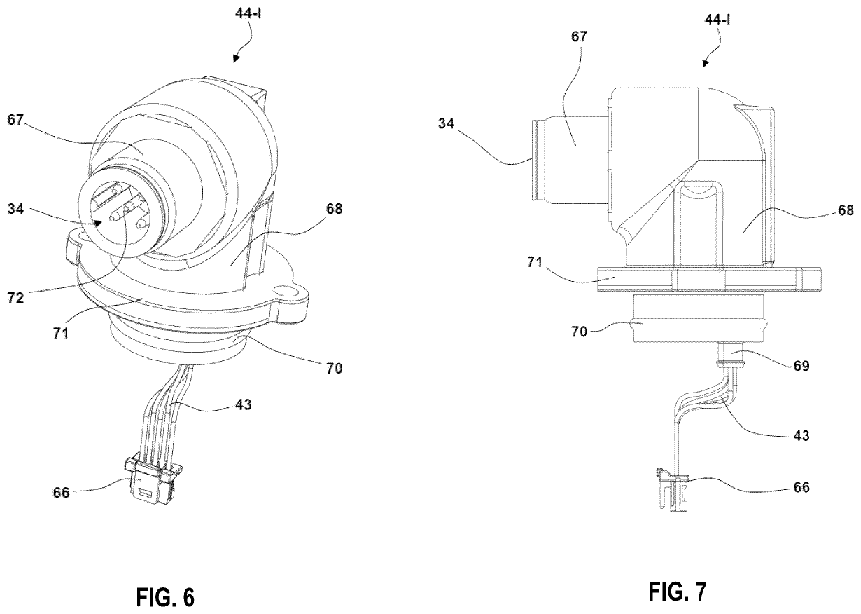

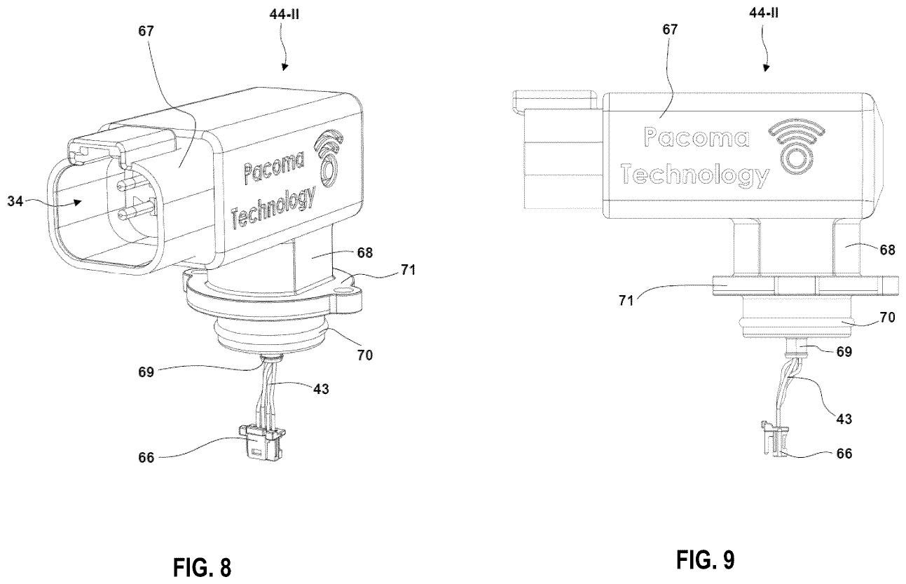

FIGS. 6 and 7 show a housing plug 44-I, where “I” here indicates that it is a housing plug of a first type (see the explanations for the first type above).

As can be seen in FIG. 7, the housing plug 44-I is L-shaped with a leg 67 and a leg 68 which is angled here at an angle of 90°. A plug receptacle is provided in the distal face of the leg 68, into which a plug 69 of the sensor cable 43 can be plugged in. In some implementations, both the plug receptacle and the plug 69 have the “pico-clasp” format.

When oriented coaxially to the longitudinal axis 53, the outer end region of the leg 68 extends into the transverse bore 27. The end region of the leg 68 can have a circumferential bead 70 or a sealing element. In the state inserted into the transverse bore 27, the bead 70 creates a frictional, elastically prestressed securing of the leg 68 in the transverse bore 27. In addition, in some implementations, a seal can be provided here.

In the exit area of the leg 68 from the housing 46 of the cylinder head 4, the leg 68 has a circumferential flange 71. The flange 71 is accommodated in a corresponding receptacle or recess in the housing 46. The flange 71 has through holes oriented parallel to the longitudinal axis 53, via which the flange 71 can be screwed to corresponding threaded holes in the housing 46. In some implementations, several holes are provided in the flange 71 and threaded holes in the housing 46, so that the housing plug 44-I can be screwed to the housing 46 in different orientations about the longitudinal axis 53.

The end region of the leg 67 forms the connecting plug 34, which enables a connecting cable to be connected. For the housing plug 44-I, the connecting plug 34 has, as shown in FIG. 6, five pins 72. In this configuration, the connection plug 34 is of the type “M12 5-pin”.

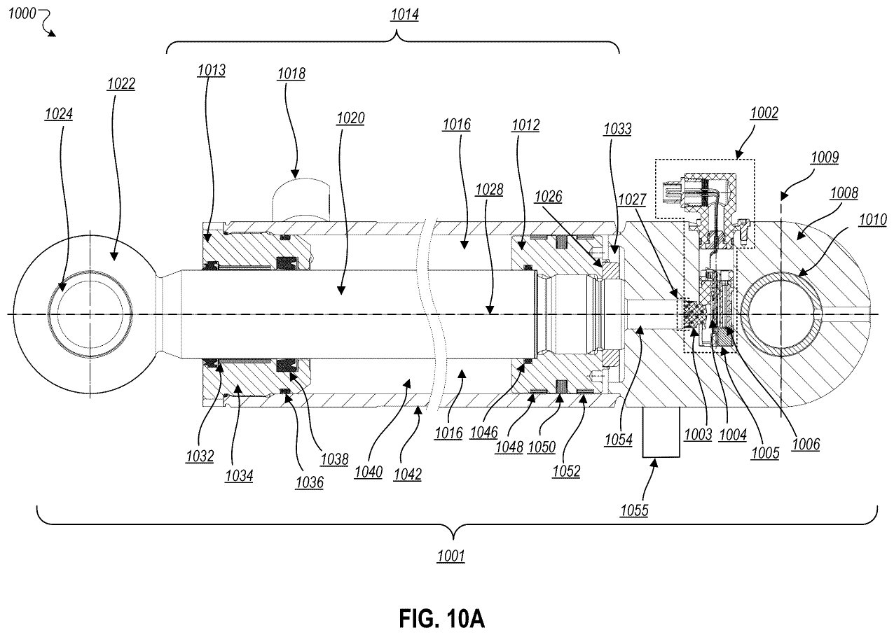

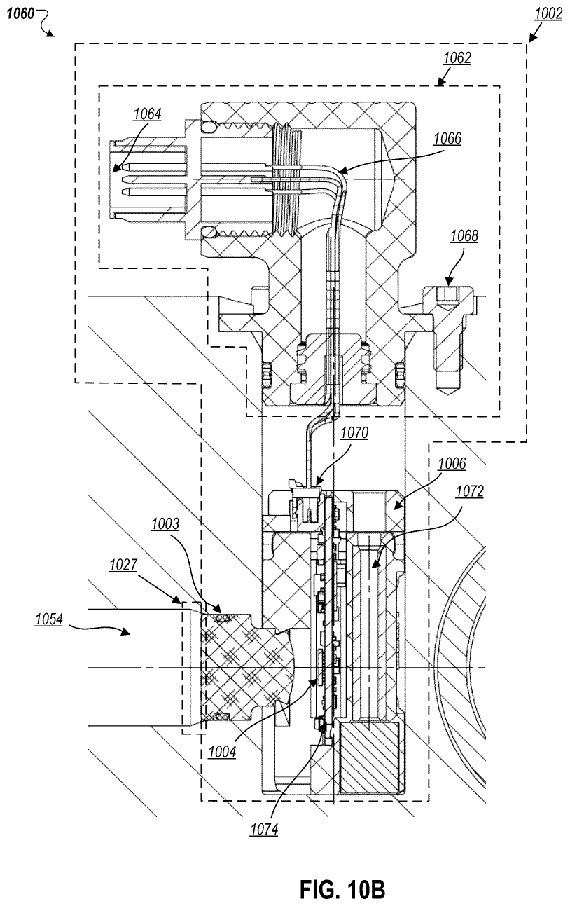

FIGS. 8 and 9 show a housing plug 44-II, where “II” here indicates that the housing plug is a housing plug of a second type, as described herein. Electronic components are integrated into the housing plug 44 in order to modify the transmitted signals from the plug 69 to the connecting plug 34.

The piston movement sensor 28 is used to directly measure the stroke of the piston 7 or the piston rod 8 within the piston-cylinder unit 1. The piston movement sensor 28 is preferably based on a non-contact measuring radar system in which the transit time between a transmitting unit and the end face of the piston 7 or the piston rod 8 and the reflected signal received at a receiving unit is evaluated. The position and/or speed of the piston 7 can then be determined from the transit time with high accuracy and robustness.

The piston-cylinder unit 1 with the integrated piston movement sensor 28 is preferably designed in accordance with protection class IP69K.

It is possible that the piston movement sensor 28 can be used to determine a stroke that is in the range of 10 mm to 2,000 mm, for example 30 mm to 1,800 mm or 40 mm to 1,600 mm. Here, for example, a resolution in the range of 0.2 mm to 4 mm, for example 0.5 to 2 mm or 0.8 to 1.5 mm, can be achieved.

Another advantage of the sealing of the sensor signal channel 26 by a sealing element or a multifunctional collimator 35 is that high hydraulic pressures, which can be, for example, 100 bar to 600 bar, do not lead to deformations, stresses and damage to the piston movement sensor 28, the sensor housing 55 and/or the electronic components of the piston movement sensor 28.

The pico-clasp connector used for the sensor cable 43 and its connection to the piston movement sensor 28 and the housing plug 44 can have five pins, which can be assigned GND, VDC, CAN LO, CAN HI and an analog signal.

The analog signal can be used to transmit a pulse width modulated signal (PWM), with the measurement signal being transmitted via pulse width modulation. Alternatively, it is possible that a voltage or a current that is proportional to the measurement signal is transmitted as an analog signal. If a PWM signal is transmitted, it can have, in some implementations, a frequency of approximately 500 Hz. The duty cycle provides information about the measured path of the piston. For example, if the piston is fully retracted, the duty cycle can be 5%, while for the fully extended state of the piston, the duty cycle can be 95%.

The piston movement sensor 28 may not only measure the stroke and/or the speed of the piston 7 or the piston rod 8. It is possible that, alternatively (or in addition), other measured variables (such as the temperature) can also be measured, transmitted and/or evaluated. Temperature measurements can be used, for example, for temperature compensation.

It is also possible that a bidirectional transmission is possible via the housing plug 44, which also allows a software update of the piston movement sensor 28 to take place and update functions to be carried out by the piston movement sensor 28.

FIGS. 10A-10B show another embodiment of a piston and cylinder unit 1001. FIG. 10A is a cross-section side view 1000 of a hydraulic cylinder 1001 (e.g., a piston and cylinder unit 1001) including a sensor assembly block 1002, including a sensor unit 1004 and a dielectric lens 1003. The sensor assembly block 1002 that further includes a dielectric lens 1003 and cylinder sensor unit 1004. The hydraulic cylinder 1001 includes a cylinder head 1008 at a first end of the hydraulic cylinder 1001 (e.g., depicted on the right-hand side of the page), and includes a piston rod eye 1022 at a second end of the hydraulic cylinder 1001 (e.g., opposite the first end of the hydraulic cylinder, on the left-hand side of the page). The cylinder head 1008 includes a bearing bushing 1010 arranged in an area of the cylinder head 1008, and the piston rod eye 1022 includes bearing bushing 1024 arranged in an area of the piston rod eye 1022. Each of the bearing bushings 1010, 1024 facilitate connections between the hydraulic cylinder 1001 and a machine, e.g., to provide motion for the machine.

FIG. 10A shows a longitudinal center axis 1028 illustrated as a dashed line across the length of the hydraulic cylinder 1001 as a reference for the insertion of components into portions of the hydraulic cylinder 1001. For example, a cylinder sensor unit 1004 can be inserted into the cylinder head 1008 by a sensor mounting bore 1005 (also referred to as a “cavity 1005”). FIG. 10A also shows a vertical axis 1009 illustrated as a dashed line substantially perpendicular to the longitudinal center axis 1028, as a reference for the insertion of components into portions of the hydraulic cylinder 1001. For example, the sensor assembly block 1002 can be inserted radially into the cavity 1005 of the cylinder head 1008.

The cylinder head 1008 is coupled to a cylinder body 1014, which further includes a cylinder housing 1042 (also referred to as “housing 1042”). The housing 1042 is configured to house the components of the cylinder body 1014, such as a piston 1012, piston rod 1020, etc. The piston 1012 is connected to a piston rod 1020, in which the piston rod eye 1022 is arranged at the second end of the hydraulic cylinder 1001. The housing 1042 can be scaled, e.g., hermetically, using a number of components (e.g., mechanical gaskets, seals, rings) to maintain pressure inside of the hydraulic cylinder 1001.

The piston 1012 effectively separates an interior of the cylinder body 1014 into a pair of pressure chambers 1016 and 1033 on either side of the piston 1012. The interior of the cylinder body 1014 can be filled with a hydraulic fluid via a connection, e.g., by port 1018. For example, pressure chamber 116 is illustrated adjacent and to the left of the piston 1012, whereas pressure chamber 133 is illustrated to the right of the piston 1012. The pressure chamber 1016 is formed in the interior of the cylinder body 1014 and surrounds the piston rod 120. Referring to ports 1018 and 1055 of the hydraulic cylinder 1001, the ports can be filled with hydraulic fluid to generate different amounts of pressure to generate motion for the piston. Port 1018 can be configured to fill the pressure chamber 1016, while port 1055 can be configured to fill the pressure chamber 1033.

For example, a hydraulic circuit (not illustrated in FIG. 10A), with a hydraulic pump and changeover valves is connected to the port 1018 and/or port 1055 to allow exchange of hydraulic fluids and generate different amounts of pressure. For example, depending the pressure generated by means of the hydraulic circuit at the port 1018 and/or port 1055, an actuating force can be generated hydraulically with both directions along the longitudinal center axis 1028, which acts on the piston 1012, and with the resulting actuating movement of the piston 1012, a change in the volume of the pressure chambers 1016 and 1033.

Although FIG. 10A shows the piston 1012 and the piston rod 1020 in a fully retracted position within the cylinder body 1014, the piston 1012 and the piston rod 1020 can also be extended by sliding along the longitudinal center axis 1028. As the piston 1012 slides along longitudinal axis 1028, the relative sizes of the pressure chambers 1016 and 1033 on either side of the piston 1012 will correspondingly change based on the position of piston 1012 within the cylinder body 1014. A rod seal 1038 and an O-ring 1036 are provided for storage and scaling at a bottom portion of the cylinder body 1014. The bottom portion of the cylinder body 1014 also includes a slide bearing 1032 to support sliding motions of the piston rod 1020. The cylinder body 1014 includes a guide bushing 1013 on a front portion of the cylinder body 1014 (e.g., left hand side of the page) to stabilize and guide the movement of the piston rod 1020 within the cylinder body 1014, by stabilizing the piston rod as it extends and retracts in the cylinder body 1014.

The piston 1012 is rotationally fixed to the piston rod 1020 and secured by means of a lock nut 1026. Furthermore, an O-ring 1046, a piston guide ring 1048, a piston seal 1050, and a further piston guide ring 1052 are arranged on the piston 1012. In this way, piston 1012, piston rod 1020, and piston rod eye 1022 form a slidable unit along axis 1028 while maintaining a seal between pressure chambers 1016, 1033. To the right of the pressure chamber 1033 (e.g., enclosed by the cylinder housing 1042, a partial chamber 1054 of the pressure chamber 1033 in the cylinder head 1008 connects the cylinder head 1008 to the cylinder body 1014. The partial chamber 1054 includes an axially extending sensor signal channel 1027, shown in FIG. 10A as part of the pressure chamber 1033 and thus exposed to hydraulic fluid. The sensor signal channel 1027 is in turn connected to the cavity 1005, which extends radially relative to the longitudinal center axis 1028 in the cylinder head 1008. The cavity 1005 extends to the outer surface of the cylinder head 1008 and can be connected to the environment by means of an unillustrated compensation hole. The sensor signal channel 1027 is adjacent to the dielectric lens 1003, to facilitate propagation of electromagnetic beams between the cylinder body 1014 and the cylinder sensor unit 1004 of the sensor assembly block 1002.

FIG. 10B is a close-up, cross-sectional view 1060 of the longitudinal section (e.g., a longitudinal portion) of the piston and cylinder unit 1001 shown in FIG. 10A. The sensor block 1002 is arranged in the cavity 1005, such that the cylinder sensor unit 1004 (also referred to as a “piston position detection unit 1004” or “sensor unit 1004”) can be used to detect the axial position of the piston 1012 and/or the piston rod 1020 in the cylinder body 1014 using high-frequency technology (e.g., using radar signals). The sensor block 1002 can include a housing for the sensor unit 1004, e.g., to secure the sensor unit 1004 in the cavity 1005.

The sensor unit 1004 can be a radar sensing unit that includes one or more radar sensors and/or emitters configured to emit radar signals into the cylinder body 1014 and detect reflected radar signals. The sensor unit 1004 sends out a high-frequency signal, which hits the end face of the piston 1012 or the piston rod 1020 through the sensor signal channel 1027 and through the partial chamber 1054 as well as through the pressure chamber 1033 and, after reflection through this end face, returns to the sensor unit 1004.

The movement of the signal, in particular the path traveled by the end face, can then be determined from the reflected signal using high-frequency technology, in particular by evaluating the transit time. For example, an electronic unit connected to or included in the sensor unit 1004 (including electronic components and software executed by these components) can carry out an evaluation of the reflected signals to determines the current position of the piston 1012 along the longitudinal center axis 1028. This determination can be conducted permanently, in defined time intervals, continuously, or at specific points in time. In some implementations, the result or a command being associated with the result is transmitted to an electronic computing unit of the working machine connected therewith—a part of which is the hydraulic cylinder 1001.

The sensor unit 1004 can be used to directly measure the stroke of the piston 1012 or the piston rod 1020 within the cylinder body 1014. The sensor unit 1004 is preferably based on a non-contact measuring radar system in which the transit time between a transmitting unit and the end face of the piston 1012 or the piston rod 1020 and the reflected signal received at a receiving unit is evaluated. The position and/or speed of the piston 1012 can then be determined from the transit time with high accuracy and robustness. For example, the sensor unit 1004 can be used to determine a stroke that is in the range of 10 mm to 2,000 mm, for example 30 mm to 1,800 mm or 40 mm to 1,600 mm. Here, for example, a radar detection resolution in the range of 0.2 mm to 4 mm, for example 0.5 to 2 mm or 0.8 to 1.5 mm, can be achieved.

The sensor block 1002 can include a sensor housing 1006 that that includes the sensor unit 1004 coupled to the dielectric lens 1003. The sensor block 1002 can be position in the cavity 1005 to form a seal that prevents the hydraulic fluid from escaping, e.g., from the partial chamber 1054 and into the sensor unit 1004. The seal can be formed between the partial chamber 1054 and the dielectric lens 1003, and between the dielectric lens 1003 and the sensor housing 1006.

The dielectric lens 1003 is configured to direct high-frequency signals in a way that improves measurement accuracy of the sensor unit 1004. The dielectric lens 1003 can be formed such that beams that were previously non-parallel beams (e.g., from divergent sources) can be made parallel to one another, e.g., converting parallel beams to non-parallel beams and vice-versa. For example, the sensor unit 1004 can transmit beams from a central point (e.g., a transmitter or transceiver of the sensor unit) to a front side of the piston 1012 and/or the piston rod 1020. The dielectric lens 1003 converts the non-parallel beams into a set of parallel beams while the beams propagate through the dielectric lens 1003, such that the beams exit through the dielectric lens substantially parallel, e.g., relative to one another. Upon the beams illuminating parts of the cylinder body 1014, the resulting return signals (e.g., including information for forming detections by the sensor unit) are reflected back into parallel beams. The dielectric lens 1003 can be configured to receive the return signals at substantially parallel beams and bundle the beams back to a central point of the sensor unit 1004, e.g., a receiver or transceiver of the sensor unit.

In some implementations, the dielectric lens 1003 can be configured (e.g., based on the material and/or shape) to serve as a filter that focuses only on high-frequency beams or substantially high-frequency beams for the sensor unit, e.g., beams that have propagated through the dielectric lens at a substantially parallel angle and to the longitudinal center axis 1028. This allows high-frequency radiation to be filtered out that does not originate, or at least does not originate directly from an end of the piston 1012 and/or piston rod 1020. A source of clutter from the receive signals can result from the fact the refraction/reflection of beams may not be ideal, e.g., beams transmitted and/or received may not occur punctually or surfaces may not be ideally flat.

The dielectric lens 1003 can be made up or have a dielectric plastic or a dielectric ceramic, polytetrafluoroethylene, polyethylene or polypropylene. The dielectric lens 1003 preferably has a dielectric constant (permittivity) greater than that of air and greater than that of the hydraulic fluid in the piston-cylinder unit. For example, the permittivity can be between 20% and 50% greater than that of the hydraulic fluid in the cylinder body 1014. The permittivity difference and the curvature of the dielectric lens are coordinated. In some implementations, the dielectric lens 1003 may be formed by the sensor block 1002 or by the sensor unit 1004 itself, although the sensor housings can be structurally separated.

FIG. 10B also illustrates a housing connector 1070 for carrying electrical signals, such as a pico-clasp plug that can be used to connect a housing plug 1062 to the sensor unit 1004, e.g., by mounting the sensor unit 1004 onto a substrate 1074 and coupling the housing connector 1070 to the substrate 1074. For example, the substrate 1074 can include one or more ports configured to receive the housing connector 1070. The substrate 1074 can include one or more electrical components mounted on a surface of the substrate 1074, embedded in the substrate 1074, etc. In some implementations, the substrate 1074 is a printed circuit board (PCB), with a number of electrical components mounted on the PCB. Examples of additional components can include various power stage components such as amplifiers, current/voltage regulators and converters, etc.

The sensor block 1002 can include the housing plug 1062 with a number of components that facilitate connections to and from a device for providing control to the hydraulic cylinder, e.g., a computing device. For example, the housing plug 1062 includes a connector plug 1064 to couple to a connector cable from a device to provide signals to the sensor unit 1004. The sensor block 1002 can include a housing connector 1070 that attaches to the sensor unit 1004 (e.g., through the housing connector 170 coupled to the PCB 174, where the sensor unit 1004 is mounted) using a number of wires 1066. In some cases, the housing connector 1070 can be coupled to the sensor unit 1004, prior to the insertion of the sensor unit 1004 into the cavity 1005. In some implementations, the housing connector 1070 and/or the connector plug 1064 is an M12 connector, although any other type of hydraulic cylinder connector configured to carry to provide signals may be utilized. The sensor block 1002 can include one or more fixing screws to affix the housing plug 1062 to the cylinder head 1008. The cylinder 1001 also includes a threaded pipe 1072, which can be used to align the position of the cavity to the sensor block 1002.

The housing plug 1062 includes a number of pins, e.g., ground, DC voltage, analog signal, high-speed bus, low-speed bus for communication to and from the sensor unit 1004 and other devices. For example, the housing plug 1062 can use an analog signal to provide pulse-width modulated pulses or voltage signals. The sensor block 1002 can include one or more fixing screws 1068 to affix the housing plug 1062 to the cylinder head 1008. The sensor block 1002 also includes a threaded pipe 1072 which can be used to align the position of the sensor housing 1006 in the cavity 1005.

Many variations and modifications may be made to the example embodiments of the piston and cylinder units 1, 1001 described in relation to FIGS. 2-9 and FIGS. 10A-10B without departing substantially from the spirit and principles of the technology disclosed in this specification. In general, the contamination detection techniques described herein can be implemented using any piston and cylinder unit that includes a radar sensing unit (e.g., the piston position detection unit 28) that transmits radar signals through a hydraulic fluid (e.g., oil) to detect a position of the piston (e.g., the piston 7) within the cylinder (e.g., the cylinder 2). All such modifications and variations are intended to be included herein within the scope of the present disclosure.

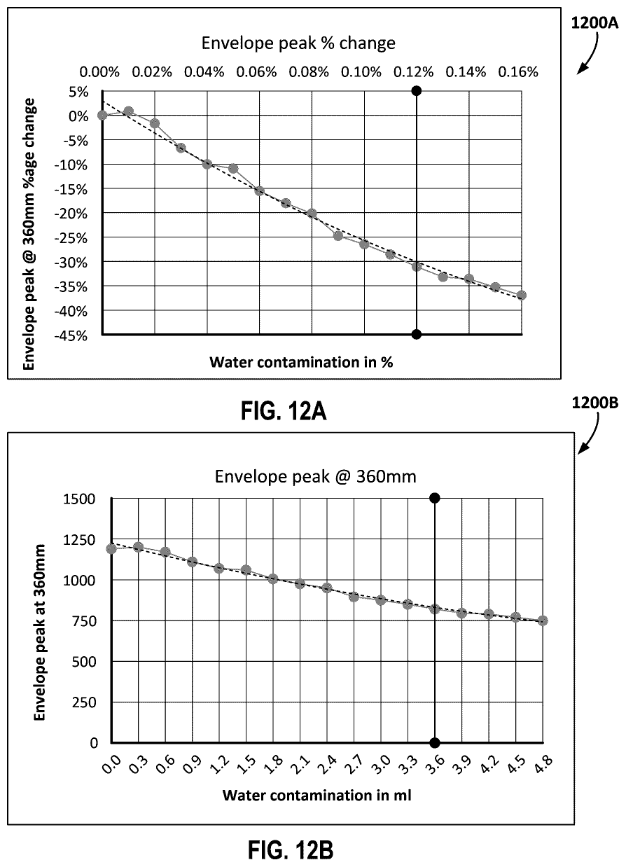

Referring now to FIGS. 11A-11C, plots 1100A, 1100B, and 1100C show example empirical data that demonstrates the relationship between measurements taken by a radar sensing unit in a cylinder and levels of water contamination in a mineral oil-based hydraulic fluid (HLP46) within the cylinder. For example, the radar sensing unit can correspond to the radar sensing unit 108 shown in FIGS. 1A and 1B, the piston position detection unit 28 shown in FIGS. 2-9, and/or the sensor assembly block 1002 shown in FIGS. 10A-10B. The cylinder can correspond to the cylinder 104 shown in FIGS. 1A and 1B, the cylinder 2 shown in FIGS. 2-9, and/or the cylinder body 1014 shown in FIGS. 10A-10B.

To produce the plots 1100A, 1100B, and 1100C, a tank connected to a piston and cylinder unit (substantially similar to the piston and cylinder unit 1 shown in FIGS. 3-9) was filled with 3000 mL of HLP46 (a mineral oil-based hydraulic fluid), and checked to ensure that no air bubbles remained trapped in the hydraulic fluid. Then, a noise and ranging calibration process was performed to calibrate the measurements captured by the piston position detection unit 28, and an envelope peak of the captured measurements was recorded with the piston at the 360 mm position (e.g., the “full stroke” position, or the fully extended position within the cylinder). Next, the piston was moved to the 0 mm position (e.g., the fully contracted position within the cylinder) and 0.3 mL of water was injected into the test cylinder using a syringe. After this water contamination was added, the piston was moved again to its max stroke position and then back to the 0 mm position ten times to ensure that the water was homogeneously distributed in the hydraulic fluid. Then, another envelope peak of the captured measurements was recorded with the piston at the 360 mm position.

Moving the piston back to the 0 mm position, the 0.3 mL water injection process, the mixing process, and the measurement recording process at the 360 mm position was repeated until a total of 4.8 mL of water contamination had been added to the hydraulic fluid. In this way, envelope peak measurements at the 360 mm piston position were obtained for various levels of water contamination ranging from no contamination to 4.8 mL (or 0.16% by volume) of water contamination.

Plot 1100A shows the measured relationship between the percentage change of the envelope peak measurement at the 360 mm position and the level of water contamination (expressed as a percentage by volume). Plot 1100B shows the measured relationship between the absolute envelope peak measurement at the 360 mm position and the level of water contamination (expressed in mL of added water). Plot 1100C shows the measured relationship between the absolute envelope peak measurement at the 360 mm position and the level of water contamination (expressed as a percentage by volume). As shown in each of the plots 1100A, 1100B, 1100C, a clear and well-defined relationship exists between the envelope peak measurement at the 360 mm position and the level of water contamination, with the envelope peak measurement decreasing as the level of water contamination increases. Thus, this empirical data demonstrates that it is possible to derive the level of water contamination in the cylinder based on the envelope peak measurements captured by a radar sensing unit (e.g., by obtaining measurements with the piston at the 360 mm full stroke position and comparing these measurements with previously obtained historical measurements).

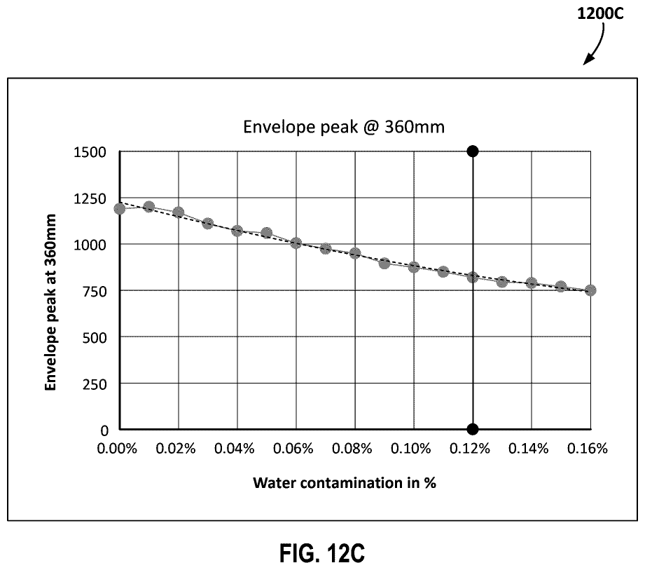

Referring now to FIGS. 12A-12C, plots 1200A, 1200B, and 1200C show example empirical data that demonstrates the relationship between measurements taken by a radar sensing unit in a cylinder and levels of water contamination in a biodegradable PAO-based hydraulic fluid (AVIA Syntofluid PE-B 50) within the cylinder. For example, the radar sensing unit can correspond to the radar sensing unit 108 shown in FIGS. 1A and 1B, the piston position detection unit 28 shown in FIGS. 2-9, and/or the sensor assembly block 1002 shown in FIGS. 10A-10B. The cylinder can correspond to the cylinder 104 shown in FIGS. 1A and 1B, the cylinder 2 shown in FIGS. 2-9, and/or the cylinder body 1014 shown in FIGS. 10A-10B.

The plots 1200A, 1200B, 1200C were produced using a similar methodology to that used to obtain the plots 1100A, 1100B, 1100C with the exception that HLP46 was replaced with AVIA Syntofluid PE-B 50 as the hydraulic fluid of interest. Plot 1200A shows the measured relationship between the percentage change of the envelope peak measurement at the 360 mm position and the level of water contamination (expressed as a percentage by volume). Plot 1200B shows the measured relationship between the absolute envelope peak measurement at the 360 mm position and the level of water contamination (expressed in mL of added water). Plot 1200C shows the measured relationship between the absolute envelope peak measurement at the 360 mm position and the level of water contamination (expressed as a percentage by volume). As shown in each of the plots 1200A, 1200B, 1200C, a clear and well-defined relationship exists between the envelope peak measurement at the 360 mm position and the level of water contamination, with the envelope peak measurement decreasing as the level of water contamination increases. Thus, this empirical data demonstrates that for biodegradable PAO-based hydraulic fluids (in addition to mineral oil-based hydraulic fluids) it is possible to derive the level of water contamination in the cylinder based on the envelope peak measurements captured by a radar sensing unit (e.g., by obtaining measurements with the piston at the 360 mm full stroke position and comparing these measurements with previously obtained historical measurements).

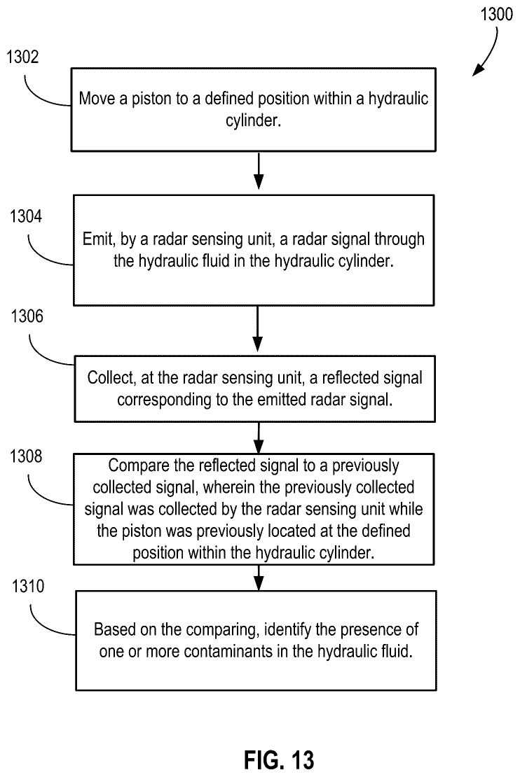

Referring now to FIG. 13, a process 1300 for monitoring a hydraulic fluid is shown and described. In some implementations, the operations of the process 1300 can be executed by a piston and cylinder unit (e.g., the piston and cylinder unit 100 shown in FIGS. 1A and 1B, the piston and cylinder unit 1 shown in FIGS. 2-9, the piston and cylinder unit 1001 shown in FIGS. 10A-10B, etc.) and/or by one or more computing devices connected to or included within the piston and cylinder unit (e.g., one or more computing devices described in further detail below in relation to FIG. 14).

Operations of the process 1300 include moving a piston to a defined position within a hydraulic cylinder (1302). For example, the piston can correspond to the piston 102 shown in FIGS. 1A and 1B, the piston 7 shown in FIGS. 2-3, or the piston 1012 shown in FIGS. 10A-10B, and the hydraulic cylinder can correspond to the cylinder 104 shown in FIGS. 1A and 1B, the cylinder 2 shown in FIGS. 2-3, or the cylinder body 1014 shown in FIGS. 10A-10B. The defined position within the hydraulic cylinder can correspond to the full stroke position (e.g., a position of maximum extension), a position of maximal contraction (e.g., the 0 mm position described above), or another defined position within the cylinder.

Operations of the process 1300 also include emitting, by a radar sensing unit, a radar signal through the hydraulic fluid in the hydraulic cylinder (1304). The radar sensing unit can correspond to, for example, the radar sensing unit 108 shown in FIGS. 1A and 1B, the piston position detection unit 28 shown in FIGS. 2-5, and/or the sensor assembly block 1002 shown in FIGS. 10A-10B. The hydraulic fluid can correspond to the uncontaminated fluid 106A shown in FIG. 1A, the contaminated fluid 106B shown in FIG. 1B, or the hydraulic fluid 29 shown in FIGS. 2-3. In some implementations, the hydraulic fluid can include a mineral oil-based hydraulic fluid or a biodegradable PAO-based hydraulic fluid (with or without contaminants).

Operations of the process 1300 also include collecting, at the radar sensing unit, a reflected signal corresponding to the emitted radar signal (step 1306) and comparing the reflected signal to a previously collected signal, wherein the previously collected signal was collected by the radar sensing unit while the piston was previously located at the defined position within the hydraulic cylinder (step 1308). For example, the previously collected signals can correspond to historical measurements that are compiled and stored in memory, as described above.

Operations of the process 1300 also include, based on the comparing, identifying the presence of one or more contaminants in the hydraulic fluid (step 1310). For example, this identification can be based on a comparison of calculated dielectric constants, a comparison of signal measurement attenuation, and/or other signal measurement characteristics, as described above. In some implementations, identifying the presence of one or more contaminants in the hydraulic fluid can include detecting a difference in a time-of-flight associated with the reflected signal and a time-of-flight associated with the previously collected signal, wherein the detected difference is greater than a threshold difference. In some implementations, identifying the presence of one or more contaminants in the hydraulic fluid can include detecting an attenuation in the reflected signal compared to the previously collected signal, wherein the attenuation is greater than a threshold attenuation level. In some implementations, identifying the presence of one or more contaminants in the hydraulic fluid can include accounting for one or more properties of the hydraulic fluid (e.g., temperature, a type of hydraulic fluid, etc.) and/or a time when the hydraulic fluid was last replaced. In some implementations, the one or more contaminants can include at least one of (i) water content in the hydraulic fluid, (ii) a presence of residues, or (iii) a presence of one or more particles in the hydraulic fluid.

Additional operations of the process 1300 can include replacing the hydraulic fluid within the hydraulic cylinder subsequent to identifying the presence of one or more contaminants in the hydraulic fluid. The process 1300 can also include providing a notification to a user or operator of the hydraulic cylinder that indicates to the user or operator that the presence of one or more contaminants in the hydraulic fluid has been identified. In some implementations, the notification to the user or operator of the hydraulic cylinder can further include an instruction to replace the hydraulic fluid within the hydraulic cylinder or to replace the hydraulic cylinder itself.

FIG. 14 shows an example of a computing device 1400 and a mobile computing device 1450 that are employed to execute implementations of the present disclosure. For example, the computing device 1400 and/or the mobile computing device 1450 can correspond to computing devices such as microcontrollers (or other computing devices) connected to or included within the radar sensing unit 108, the piston position detection unit 28, the sensor block assembly 1002, and/or the piston and cylinder units 1, 100, 1001. The computing device 1400 and/or the mobile computing device 1450 can also be employed to execute one or more steps of the process 1300 including steps 1308 and 1310. In some implementations, the computing device 1400 and/or the 1450 can also be employed to perform supporting steps to the process 1300 such as controlling the movement of the piston to the defined position within the hydraulic cylinder, controlling the radar sensing unit to emit a radar signal, and/or controlling the radar sensing unit to collect the reflected signal corresponding to the emitted radar signal. In some implementations, the computing device 1400 and/or the 1450 can implement computer software such as the examples of computer software described throughout this specification (e.g., the computer software described in relation to FIGS. 1A and 1B above).

In some implementations, the computing devices connected to or included within the radar sensing unit 108, the piston position detection unit 28, and/or the piston and cylinder units 1, 100, 1001 can include a singular computing device 1400 or mobile computing device 1450. However, in other implementations, the computing devices connected to or included within the radar sensing unit 108, the piston position detection unit 28, the sensor block assembly 1002 and/or the piston and cylinder units 1, 100, 1001 can include multiple computing devices 1400 and/or mobile computing devices 1450 that jointly perform the operations disclosed above in a distributed manner (e.g., via cloud computing). Moreover, in some implementations, computing tasks performed by the computing devices connected to or included within the radar sensing unit 108, the piston position detection unit 28, the sensor block assembly 1002, and/or the piston and cylinder units 1, 100, 1001 can be redistributed amongst one another without limitation, unless otherwise stated herein.

The computing device 1400 is intended to represent various forms of digital computers, such as laptops, desktops, workstations, personal digital assistants, servers, blade servers, mainframes, and other appropriate computers. The mobile computing device 1450 is intended to represent various forms of mobile devices, such as personal digital assistants, cellular telephones, smart-phones, AR devices, and other similar computing devices. The components shown here, their connections and relationships, and their functions, are meant to be examples only, and are not meant to be limiting.

The computing device 1400 includes a processor 1402, a memory 1404, a storage device 1406, a high-speed interface 1408, and a low-speed interface 1412. In some implementations, the high-speed interface 1408 connects to the memory 1404 and multiple high-speed expansion ports 1410. In some implementations, the low-speed interface 1412 connects to a low-speed expansion port 1414 and the storage device 1406. Each of the processor 1402, the memory 1404, the storage device 1406, the high-speed interface 1408, the high-speed expansion ports 1410, and the low-speed interface 1412, are interconnected using various buses, and may be mounted on a common motherboard or in other manners as appropriate. The processor 1402 can process instructions for execution within the computing device 1400, including instructions stored in the memory 1404 and/or on the storage device 1406 to display graphical information for a graphical user interface (GUI) on an external input/output device, such as a display 1416 coupled to the high-speed interface 1408. In other implementations, multiple processors and/or multiple buses may be used, as appropriate, along with multiple memories and types of memory. In addition, multiple computing devices may be connected, with each device providing portions of the necessary operations (e.g., as a server bank, a group of blade servers, or a multi-processor system).

The memory 1404 stores information within the computing device 1400. In some implementations, the memory 1404 is a volatile memory unit or units. In some implementations, the memory 1404 is a non-volatile memory unit or units. The memory 1404 may also be another form of a computer-readable medium, such as a magnetic or optical disk.

The storage device 1406 is capable of providing mass storage for the computing device 1400. In some implementations, the storage device 1406 may be or include a computer-readable medium, such as a floppy disk device, a hard disk device, an optical disk device, a tape device, a flash memory, or other similar solid-state memory device, or an array of devices, including devices in a storage area network or other configurations. Instructions can be stored in an information carrier. The instructions, when executed by one or more processing devices, such as processor 1402, perform one or more methods, such as those described above. The instructions can also be stored by one or more storage devices, such as computer-readable or machine-readable mediums, such as the memory 1404, the storage device 1406, or memory on the processor 1402.

The high-speed interface 1408 manages bandwidth-intensive operations for the computing device 1400, while the low-speed interface 1412 manages lower bandwidth-intensive operations. Such allocation of functions is an example only. In some implementations, the high-speed interface 1408 is coupled to the memory 1404, the display 1416 (e.g., through a graphics processor or accelerator), and to the high-speed expansion ports 1410, which may accept various expansion cards. In the implementation, the low-speed interface 1412 is coupled to the storage device 1406 and the low-speed expansion port 1414. The low-speed expansion port 1414, which may include various communication ports (e.g., Universal Serial Bus (USB), Bluetooth, Ethernet, wireless Ethernet) may be coupled to one or more input/output devices. Such input/output devices may include a scanner, a printing device, or a keyboard or mouse. The input/output devices may also be coupled to the low-speed expansion port 1414 through a network adapter. Such network input/output devices may include, for example, a switch or router.

The computing device 1400 may be implemented in a number of different forms, as shown in FIG. 14. For example, it may be implemented as a standard server 1420, or multiple times in a group of such servers. In addition, it may be implemented in a personal computer such as a laptop computer 1422. It may also be implemented as part of a rack server system 1424. Alternatively, components from the computing device 1400 may be combined with other components in a mobile device, such as a mobile computing device 1450. Each of such devices may contain one or more of the computing device 1400 and the mobile computing device 1450, and an entire system may be made up of multiple computing devices communicating with each other.

The mobile computing device 1450 includes a processor 1452; a memory 1464; an input/output device, such as a display 1454; a communication interface 1466; and a transceiver 1468; among other components. The mobile computing device 1450 may also be provided with a storage device, such as a micro-drive or other device, to provide additional storage. Each of the processor 1452, the memory 1464, the display 1454, the communication interface 1466, and the transceiver 1468, are interconnected using various buses, and several of the components may be mounted on a common motherboard or in other manners as appropriate. In some implementations, the mobile computing device 1450 may include a camera device(s).

The processor 1452 can execute instructions within the mobile computing device 1450, including instructions stored in the memory 1464. The processor 1452 may be implemented as a chipset of chips that include separate and multiple analog and digital processors. For example, the processor 1452 may be a Complex Instruction Set Computers (CISC) processor, a Reduced Instruction Set Computer (RISC) processor, or a Minimal Instruction Set Computer (MISC) processor. The processor 1452 may provide, for example, for coordination of the other components of the mobile computing device 1450, such as control of user interfaces (UIs), applications run by the mobile computing device 1450, and/or wireless communication by the mobile computing device 1450.

The processor 1452 may communicate with a user through a control interface 1458 and a display interface 1456 coupled to the display 1454. The display 1454 may be, for example, a Thin-Film-Transistor Liquid Crystal Display (TFT) display, an Organic Light Emitting Diode (OLED) display, or other appropriate display technology. The display interface 1456 may include appropriate circuitry for driving the display 1454 to present graphical and other information to a user. The control interface 1458 may receive commands from a user and convert them for submission to the processor 1452. In addition, an external interface 1462 may provide communication with the processor 1452, so as to enable near arca communication of the mobile computing device 1450 with other devices. The external interface 1462 may provide, for example, for wired communication in some implementations, or for wireless communication in other implementations, and multiple interfaces may also be used.

The memory 1464 stores information within the mobile computing device 1450. The memory 1464 can be implemented as one or more of a computer-readable medium or media, a volatile memory unit or units, or a non-volatile memory unit or units. An expansion memory 1474 may also be provided and connected to the mobile computing device 1450 through an expansion interface 1472, which may include, for example, a Single in Line Memory Module (SIMM) card interface. The expansion memory 1474 may provide extra storage space for the mobile computing device 1450, or may also store applications or other information for the mobile computing device 1450. Specifically, the expansion memory 1474 may include instructions to carry out or supplement the processes described above, and may include secure information also. Thus, for example, the expansion memory 1474 may be provided as a security module for the mobile computing device 1450, and may be programmed with instructions that permit secure use of the mobile computing device 1450. In addition, secure applications may be provided via the SIMM cards, along with additional information, such as placing identifying information on the SIMM card in a non-hackable manner.

The memory may include, for example, flash memory and/or non-volatile random access memory (NVRAM), as discussed below. In some implementations, instructions are stored in an information carrier. The instructions, when executed by one or more processing devices, such as processor 1452, perform one or more methods, such as those described above. The instructions can also be stored by one or more storage devices, such as one or more computer-readable or machine-readable mediums, such as the memory 1464, the expansion memory 1474, or memory on the processor 1452. In some implementations, the instructions can be received in a propagated signal, such as, over the transceiver 1468 or the external interface 1462.

The mobile computing device 1450 may communicate wirelessly through the communication interface 1466, which may include digital signal processing circuitry where necessary. The communication interface 1466 may provide for communications under various modes or protocols, such as Global System for Mobile communications (GSM) voice calls, Short Message Service (SMS), Enhanced Messaging Service (EMS), Multimedia Messaging Service (MMS) messaging, code division multiple access (CDMA), time division multiple access (TDMA), Personal Digital Cellular (PDC), Wideband Code Division Multiple Access (WCDMA), CDMA2000, General Packet Radio Service (GPRS). Such communication may occur, for example, through the transceiver 1468 using a radio frequency. In addition, short-range communication, such as using a Bluetooth or Wi-Fi, may occur. In addition, a Global Positioning System (GPS) receiver module 1470 may provide additional navigation-and location-related wireless data to the mobile computing device 1450, which may be used as appropriate by applications running on the mobile computing device 1450.

The mobile computing device 1450 may also communicate audibly using an audio codec 1460, which may receive spoken information from a user and convert it to usable digital information. The audio codec 1460 may likewise generate audible sound for a user, such as through a speaker, e.g., in a handset of the mobile computing device 1450. Such sound may include sound from voice telephone calls, may include recorded sound (e.g., voice messages, music files, etc.) and may also include sound generated by applications operating on the mobile computing device 1450.

The mobile computing device 1450 may be implemented in a number of different forms, as shown in FIG. 14. For example, it may be implemented a phone device 1480, a personal digital assistant 1482, and a tablet device (not shown). The mobile computing device 1450 may also be implemented as a component of a smart-phone, AR device, or other similar mobile device.

Computing device 1400 and/or 1450 can also include USB flash drives. The USB flash drives may store operating systems and other applications. The USB flash drives can include input/output components, such as a wireless transmitter or USB connector that may be inserted into a USB port of another computing device.

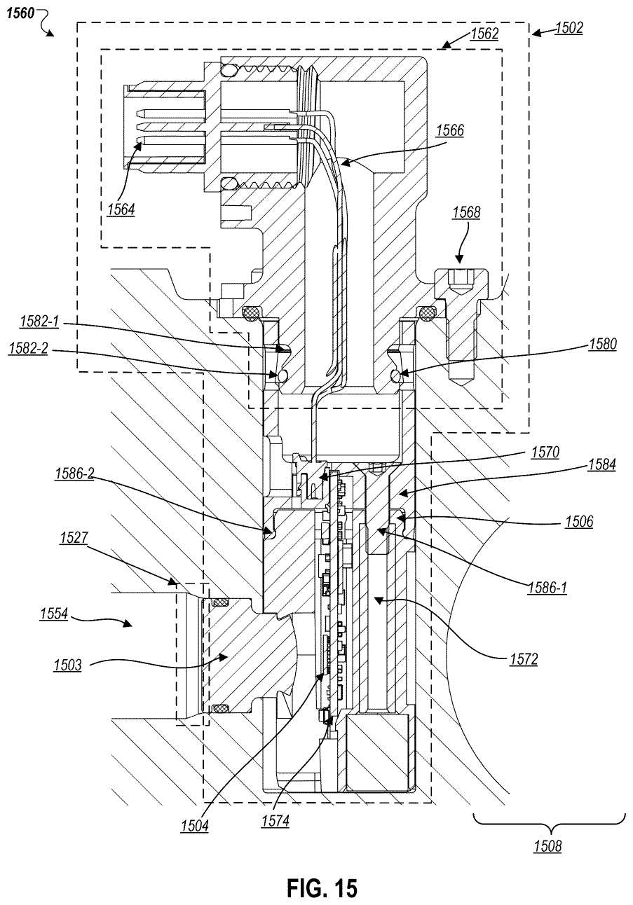

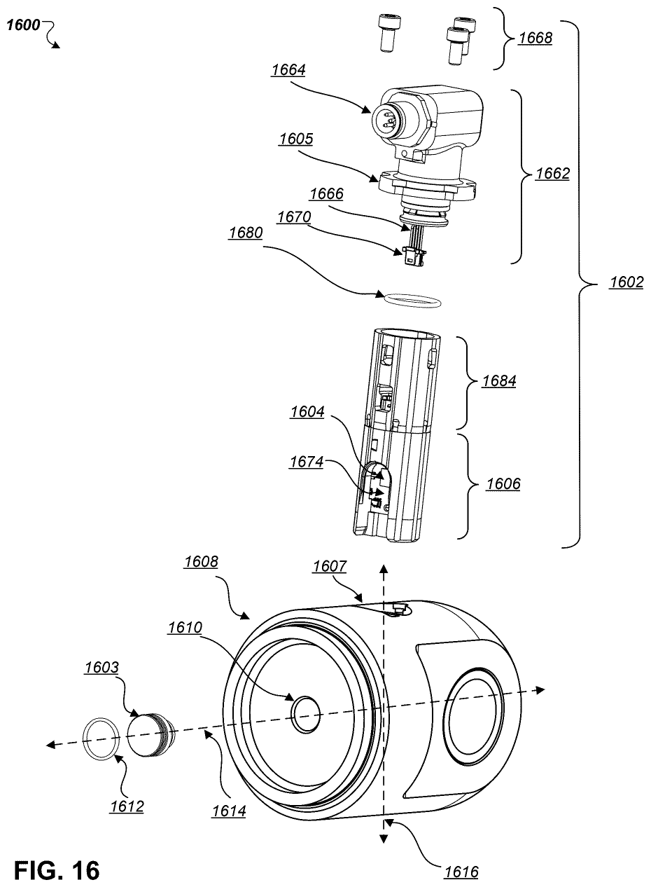

FIG. 15 is a cross-sectional view 1560 of a sensor assembly block 1502 (also referred to as a “sensor block 1502”) of a hydraulic cylinder. The sensor block 1502 is arranged in a cavity of the hydraulic cylinder and includes a cylinder sensor unit 1504. The sensor unit 1504 can be used to detect the position of a piston and/or the piston rod along a length of a cylinder body of a hydraulic cylinder using high-frequency electromagnetic waves (e.g., using radar signals). As described in reference to FIG. 16 below, the sensor block 1502 can include a housing for the sensor unit 1504, e.g., to secure the sensor unit 1504 in a cavity of the hydraulic cylinder.

The sensor unit 1504 can be a radar sensing unit that includes radar sensors and/or emitters to emit radar signals into the cylinder body and detect reflected radar signals. Movement of the piston can be determined based on the reflected signal using high-frequency technology, such as evaluating the transit time of radar signals reflected back to the sensor. The reflected signals can be used to determine the current position of a piston along the longitudinal axis of the hydraulic cylinder, e.g., at a single time instance, periodically, continuously, or at specific points in time.

The sensor block 1502 can include a sensor housing 1506 that includes the sensor unit 1504 coupled to a dielectric lens 1503. The sensor block 1502 can be positioned in a cavity of the hydraulic cylinder to form a seal that prevents hydraulic fluid from escaping, e.g., from a partial chamber 1554 of the hydraulic cylinder into the sensor unit 1504. The seal can be formed between the partial chamber 1554 and the dielectric lens 1503, and between the dielectric lens 1503 and the sensor housing 1506. The partial chamber 1554 also includes an axially extending sensor signal channel 1527.

The dielectric lens 1503 is shaped and positioned so as to direct high-frequency signals toward the sensor unit 1504. The dielectric lens 1503 can be configured (e.g., based on the material and/or shape) to serve as a filter that focuses on a target range of beams, such as high-frequency beams or substantially high-frequency beams for the sensor unit.

FIG. 15 also illustrates a housing connector 1570 for carrying electrical signals through wires 1566. The housing connector 1570 can be a pico-clasp plug that connects a housing plug 1562 to the sensor unit 1504, e.g., by mounting the sensor unit 1504 onto a substrate 1574 and coupling the housing connector 1570 to the substrate 1574. For example, the substrate 1574 can include one or more ports configured to receive the housing connector 1570. The substrate 1574 can include one or more electrical components mounted on a surface of the substrate 1574, embedded in the substrate 1574, etc.

The sensor block 1502 can include the housing plug 1562 with a number of components that facilitate connections to and from a device for providing control to the hydraulic cylinder, e.g., a computing device, e.g., through a connector plug 1564 to transmit and receive signals between a computing and the sensor unit 1504. The sensor block 1502 can include a housing connector 1570 that attaches to the sensor unit 1504 (e.g., through the housing connector 1570 coupled to the substrate 1574, where the sensor unit 1504 is mounted). In some cases, the housing connector 1570 can be coupled to the sensor unit 1504, prior to the insertion of the sensor unit 1504 into the cavity. In some implementations, the connector plug 1564 is an M12 connector, although any other type of hydraulic cylinder connector configured to carry to provide signals may be utilized.

The housing plug 1562 includes a number of pins for communication to and from the sensor unit 1504 and other devices. The sensor block 1502 can include one or more fixing screws 1568 to affix the housing plug 1562 to a cylinder head. Although FIG. 15 depicts a close-up view of the sensor block 1502 with the cylinder head 1508, the cylinder head 1508 can include other examples of cylinder heads such as the cylinder head 1608 described in reference to FIG. 16 below. The sensor block 1502 also includes a threaded pipe 1572 which can be used to align the position of the sensor housing 1504 in a cavity of the hydraulic cylinder.

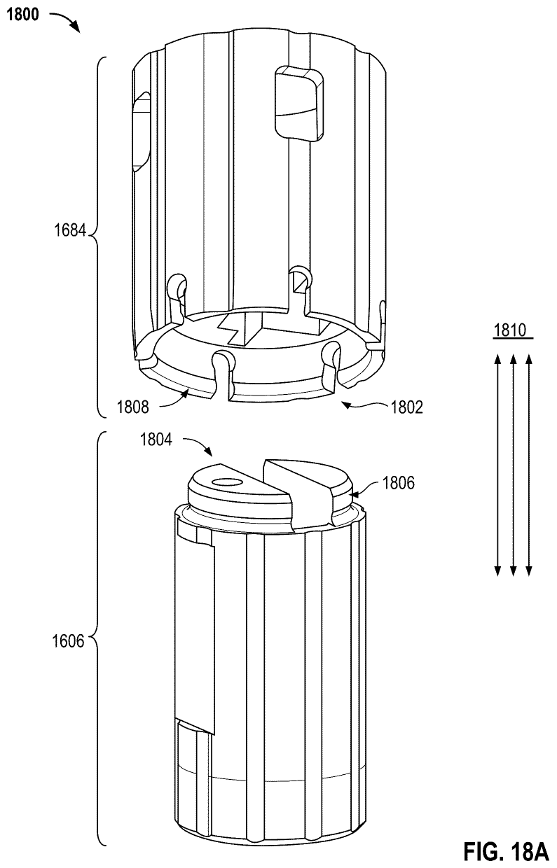

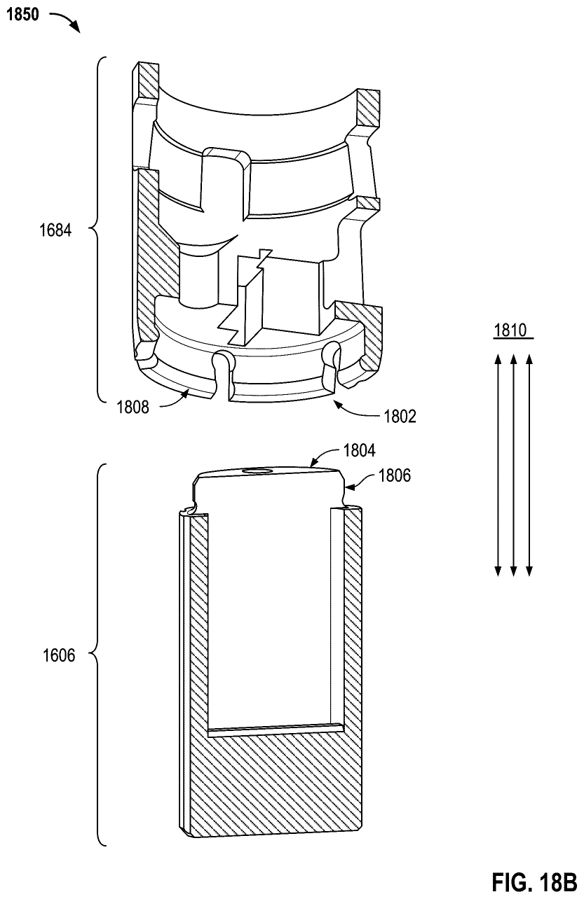

The sensor block 1502 can include an interconnection spacer 1584 that couples the sensor housing 1506 to the housing plug 1562. As described in reference to FIGS. 18A and 18B below, the interconnection spacer 1584 (“also referred to as spacer 1584”) can include a top portion to couple to the housing plug 1562 and a bottom portion to couple to the sensor housing 1506. Referring to a bottom portion of spacer 1584, the spacer 1584 attaches to the sensor housing 1506 by attachment mechanisms 1586-1 and 1586-2, which can be a latch, screw, or another type of mechanical attachment. For example, the attachment mechanism 1586-1 can be a latch and the attachment mechanism 1586-2 can be a screw. In some implementations, the attachment mechanism 1586-2 can include one or more snap features to connect the spacer 1584 to the sensor housing 1506.

The attachment mechanisms 1586-1 and 1586-2 can be configured to couple the spacer 1584 to the sensor housing 1506. At a top portion of the spacer 1584, the spacer 1584 can be retained by the housing plug 1562 by a retaining mechanism 1580, such an o-ring or another type of mechanical gasket. By coupling the sensor housing 1506 to the housing plug 1562 by the spacer 1584, the sensor block 1502 can be an assembly of three components that are mechanically connected to each other, e.g., to form a single complete assembly that provides improved rigidity for the sensor block 1502. For example, the sensor block can include a wire assembly without a structural support between the housing plug 1562 and the sensor housing. The spacer 1584 can be a mechanical component that maintains a distance between two or more objects in the assembly.

The sensor block 1502 with the spacer 1584 provides improved mechanical rigidity and stability relative to the sensor block 302. The spacer 1584 can provide a mechanical interface between the sensor housing 1506 and the housing plug 1562, to mechanically secure the sensor housing 1506 to the housing plug 1562. The spacer 1584 can provide improved robustness, by reducing or preventing pull stress on the interconnection wires 1566, as the housing plug 1562 is mechanically fixed to the sensor housing 1506 by the spacer 1584. The spacer 1584 can also provide improved efficiency during assembly and disassembly, as the entire cartridge (e.g., sensor housing 1506, spacer 1584, and housing plug 1562) can be connected as a single assembly. A single assembly provides easier installation compared to installation of a separated sensor housing 1506 and housing plug 1562. A single assembly also improves reliability and durability through the improved rigidity, e.g., to mitigate vibrational fluctuations experienced by the hydraulic cylinder during operation.

Referring to the retaining mechanism 1580, the housing plug 1562 includes an upper slot 1582-1 and a lower slot 1582-2 (collectively “slots 1582”) for positioning the retaining mechanism 1580. Examples of slots can include grooves or recesses where the retaining mechanism 1580 can be placed. For example, the retaining mechanism 1580 in upper slot 1582-1 provides assembly of the spacer 1584 to the housing plug 1562, e.g., inserting a top portion of the spacer 1584 to a bottom portion of the housing plug 1562. In the lower slot 1582-2, the retaining mechanism 1580 can be configured to retain the spacer 1584 to the housing plug 1562. While in the lower slot 1582-2, the retaining mechanism 1580 can securely retain the spacer 1584 to the housing plug 1562, e.g., to allow for insertion or engagement in the upper slot 1582-1 and to interlock to prevent removability of the spacer 1584 in the lower slot 1582-2. In the lower slot 1582-2, the spacer 1584 can allow for the sensor block 1502 to be rotated, e.g., to orient the connector plug 1564, while retaining the spacer 1584 in the sensor housing 1506. Although FIG. 15 depicts a pair of slots, the spacer 1584 can include any number of slots.

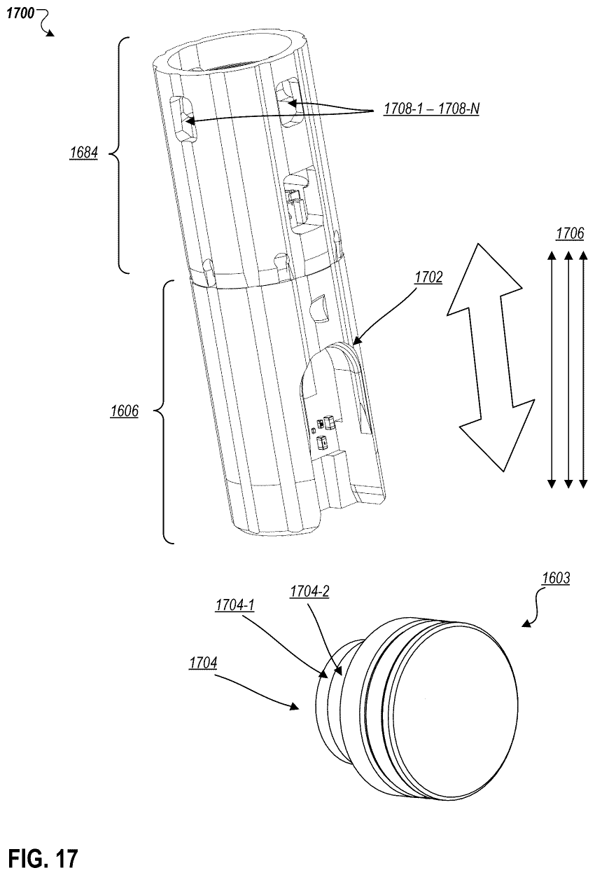

A spacer 1584 can be any length and the length of the spacer can be based on a diameter of the hydraulic cylinder. In some implementations, the length of a spacer 1584 can be approximately 3 inches. The spacer 1584 can also be a longer length for a hydraulic cylinder with a relatively large diameter, or a shorter length for a hydraulic cylinder with a relatively small diameter. The retaining mechanism 1580 can be adjusted from one slot to another, e.g., slot 1582-1 to 1582-2 or vice versa, using a tool to mechanically adjust the position by pulling the retaining mechanism 1580. As described in reference to FIG. 17 below, the spacer 1584 includes one or more openings that allow for adjustment of the retaining mechanism 1580.

FIG. 16 is an exploded view 1600 of the sensor assembly block 1602 (also referred to as “sensor block 1602”), which is an example of the sensor block 1502 of FIG. 15. The exploded view 1600 depicts the sensor block 1602 with a cylinder head 1608. The exploded view 1600 shows the sensor block 1602 having housing plug 1662, which can be affixed to cylinder head 1608 by one or more fixing screws 1668. For example, the housing plug 1662 can include a plate 1605 with a number of openings disposed through a thickness of the plate 1605. Similar to plate 305, the plate 1605 can be formed from the same body of the housing plug 1662 but can also be an additional component attached to the housing plug 1662. Each fixing screw 1668 can be disposed in an opening of the plate 1605 to secure the housing plug 1662 to the cylinder head 1608, e.g., by placing the fixing screw 1668 into an opening disposed in a surface of the cylinder head 1608.

The cylinder head 1608 also includes a sensor unit opening 1607 (also referred to as a “sensor unit cavity 1607”), which is an opening that extends through a wall of the cylinder head 1608, e.g., to form a cavity, to allow for insertion of a sensor housing unit 1606. The sensor housing unit 1606 depicted in FIG. 16 can be implemented without a cap covering a top portion of the sensor housing unit 1606. An example illustration of the sensor housing unit 1606 without a cap is depicted and described in reference to FIG. 18A below. The sensor housing unit 1606 can include a sensor unit 1604. The sensor unit 1604 is mounted on a substrate 1674. The sensor housing unit 1606 can be coupled to the housing plug 1662 by the housing connector 1670.

A housing connector 1670 can be configured to attach the sensor housing unit 1606 to the housing plug 1662 by wires configured to communicate signals between the sensor unit 1604 and a data port of the housing plug 1662, e.g., to provide signals to a computing device, machine, or some combination thereof, coupled to the hydraulic cylinder. The sensor housing unit 1606 includes a sensor unit 1604. The housing connector 1670 can be used to couple the sensor housing unit 1606 to the housing plug 1662 prior to the insertion of the sensor housing unit 1606 into a cavity of the cylinder head 1608.

As illustrated in FIG. 16, the housing plug 1662 can include a number of grooves and/or corresponding O-rings to form a seal between the housing plug 1662 and the sensor unit opening 1607, by placing the grooves (optionally including O-rings) into the sensor unit opening 1607. A seal between sensor unit opening 1607 and the housing plug 1662, can prevent water, dirt, and other particulates from entering the interior of the cylinder head 1608. In some cases, the housing plug 1662 can include a threaded surface (e.g., a number of threads) to facilitate a connection between the housing plug 1662 and the sensor unit opening 1607. In this implementation, the interior of the cylinder head 1608 can include a corresponding threaded surface that below the sensor unit opening 1607 to be coupled to the thread surface of the housing plug 1662.

Similar to the sensor block 1502 described above in reference FIG. 15, the exploded view 1600 depicts the sensor block 1602 having an interconnection spacer 1684 that couples the sensor housing 1606 to the housing plug 1662, e.g., similar to spacer 1584 described in reference to FIG. 15. The interconnection spacer 1684 (“also referred to as spacer 1684”) can include a top portion to couple to the housing plug 1662 and a bottom portion to couple to the sensor housing 1606. Referring to a bottom portion of spacer 1684, the spacer 1684 attaches to the sensor housing 1606 by attachment mechanisms, e.g., a latch, screw, snap feature, or another type of mechanical attachment.

At a top portion of the spacer 1684, the spacer 1684 can be retained by the housing plug 1662 by a retaining mechanism 1680, such an O-ring or another type of mechanical gasket. By coupling the sensor housing 1606 to the housing plug 1662 by the spacer 1684, the sensor block 1602 can be an assembly of three components that are mechanically connected to each other, e.g., to form a single complete assembly that provides improved rigidity, durability, and easy of assembly/disassembly.

Similar to sensor block 1502 described in reference to FIG. 15, the sensor block 1602 As described in reference to FIG. 15, the spacer 1684 of the sensor block 1602 provides a mechanical interface between the sensor housing 1606 and the housing plug 1662, to mechanically secure the sensor housing 1606 to the housing plug 1662. The spacer 1684 can provide improved robustness, by reducing or preventing pull stress on the interconnection wires 1666, as the housing plug 1662 is mechanically fixed to the sensor housing 1606 by the spacer 1684. The spacer 1684 can also provide improved efficiency during assembly and disassembly, as the entire cartridge (e.g., sensor housing 1606, spacer 1684, and housing plug 1662) can be connected as a single assembly.

The retaining mechanism 1680 can also be positioned inside of the spacer 1684, e.g., such as a slot of the housing plug 1662, to retain the spacer 1684 to the housing plug 1662 to allow for interlocking that prevents removal of the spacer 1684 from the housing plug 1662. The spacer 1684 can allow for the sensor block 1602 to be rotated, e.g., to orient the connector plug 1664, while retaining the spacer 1684 in the sensor housing 1606. For example, the retaining mechanism 1680 can be inserted into the spacer 1684.

The exploded view 1600 also shows the cylinder head 1608 having a dielectric lens opening 1610 (also referred to as a “lens cavity 1610”), as an opening disposed through a thickness of the cylinder head 1608. The opening 1610 can also be referred to as a bore 1610. For example, the cylinder head 1608 can have an opening that is partially disposed through a front surface of the cylinder head 1608 to form a bore. The opening of the bore 1610 allows for the dielectric lens 1603 to be inserted. The bore 1610 can extend between a cavity (e.g., sensor unit opening 1607) of the cylinder head 1608 and the interior of the cylinder body along the longitudinal axis, e.g., axis 1614 shown in FIG. 16. Axis 1614 can be a longitudinal center axis. FIG. 16 also shows an axis 1616 substantially perpendicular to axis 1614, in which axis 1616 shows the sensor unit opening 1607 extending vertically in the cylinder head 1608. The sensor block 1602, including the sensor housing unit 1606 coupled to the spacer 1684 (further couples to the housing plug 1662) the can be inserted into the cavity 1607.

The dielectric lens 1603 can be coupled to the sensor housing unit 1606, to allow for propagation of beams between (e.g., to and from) the sensor unit 1604 of the sensor housing unit 1606 and the dielectric lens 1603. The exploded view 1600 also shows an O-ring 1612 configured to form a seal between the dielectric lens 1603 and the bore 1610 of the cylinder head 1608, e.g., to help stabilize the dielectric lens and reduce the effects of vibrations in signal data quality. The O-ring 1612 also provides a seal between the sensor unit opening 1607 (e.g., in addition to sensor housing unit 1606) and a partial chamber of the hydraulic cylinder.

FIG. 17 is a close up view 1700 of a sensor housing unit, spacer, and a dielectric lens for the sensor assembly block of FIGS. 15 and 16. The close up view 1700 shows the sensor housing unit 1606 coupled to the spacer 1684, with the dielectric lens 1603 for the sensor assembly block (e.g., sensor assembly block 1502 of FIG. 15 and assembly block 1602 of FIG. 16). The view 1700 shows the sensor housing unit 1606 and the dielectric lens 1603 each containing a shape that allows for the sensor housing unit 1606 to receive the dielectric lens 1603 by sliding the dielectric lens into an opening of the sensor housing unit 1606. The dielectric lens 1603 includes circumferential grooves 1704, which are shown in FIG. 17 with a round shape. The sensor housing unit 1606 includes an opening 1702 with recesses that are configured to receive the circumferential grooves 1704 of the dielectric lens 303. Each recess of the opening 1702 of the sensor housing unit 1606 can contain a shape that fits to the circumferential grooves 1704 of the dielectric lens 303. For example, the recesses of the opening 1702 of the sensor housing unit 1606 can have a round shape that allows for the round shape of the circumferential grooves 1704 to fit into the recesses of the opening 1702. The dielectric lens 1603 can be coupled to the sensor housing unit 1606 by sliding the dielectric lens 1603 into the opening 1702 of the sensor housing unit 1606, e.g., along a direction 1706 shown in FIG. 17. In this way, the circumferential grooves 1704 of the dielectric lens 1603 can slide into the recesses of the opening 1702 of the sensor housing unit 1606.

The view 1700 also depicts the spacer 1684 having one or more openings 1708-1 through 1708-N (collectively “openings 1708”). As described in reference to FIG. 15 above, a spacer, e.g., spacer 1684, can include openings 1708 to allow for adjustments to a retaining mechanism 1680, e.g., to adjust the position of the retaining mechanism 1680 from one slot to another slot, such as grooves of an internal portion of the spacer 1684. The retaining mechanism 1680 can be adjusted from one slot to another slot using a tool to mechanically adjust the position of the retaining mechanism 1680, e.g., by pulling the retaining mechanism 1680 upward or downward between two different slots or grooves.

FIG. 18A shows a close-up view of the spacer 1684 and a top view of sensor housing unit 1606, of FIGS. 15, 16, and 17. The close-up view 1800 depicts the spacer 1684 having a bottom portion 1802 and the sensor housing unit 1606 having a top portion 1804. The top portion 1804 of the sensor housing unit 1606 can include one or more protrusions 1806, such as a groove, notch, or ridge. The top portion 1804 can be an example of a sensor housing unit without a cap. The top portion 1804 of the sensor housing unit 1606 can be inserted into the bottom portion 1802, e.g., to couple the sensor housing unit 1606 to the spacer 1684. In some implementations, the protrusions 1806 of the sensor housing unit 1606 can be inserted into the bottom portion 1802 of the spacer 1684, such as in a vertical direction 1810 to retain the sensor housing unit 1606 in the spacer 1684. For example, the top portion 1804 of the sensor housing unit 1606 can be inserted into the bottom portion 1802 of the spacer 1684 by moving the sensor housing 1606 upward and into the bottom portion 1802. As another example, the bottom portion 1802 of the spacer 1684 can be placed over and moving downward onto the top portion 1804 of the sensor housing unit 1606. In some implementations, the top portion 1804 of the sensor housing 1606 can be placed over and move into the bottom portion 1802 of the spacer 1684.

The spacer 1684 can include a number of attachment mechanisms 1808, e.g., latches, configured to engage with the protrusions 1806, e.g., to retain the sensor housing unit 1606. The attachment mechanisms 1808 can be an example of attachment mechanisms 1586-1 and 1586-2, as described in reference to FIG. 15 above. Examples of latches can include snap-fit latches and sliding latches. For example, a latch can be configured to temporarily bend, e.g., while connected the spacer 1684 to the sensor housing unit 1606, and snap into a locking position. The attachment mechanisms 1808 can include screws to retain the sensor housing unit 1606, e.g., by the protrusions 1806 of the sensor housing unit 1606.

FIG. 18B shows a cross-sectional view of the spacer and the sensor housing unit of FIG. 18A. The cross-sectional view 1850 shows the bottom portion 1802 of the spacer 1684 and the top portion 1804 of the sensor housing unit 1606. Similar to FIG. 18A above, the top portion 1804 can include protrusions 1806 of the sensor housing unit 1606 can be inserted into the bottom portion 1802 of the spacer 1684, e.g., to couple the sensor housing unit 1606 to the spacer 1684. Similarly, the protrusions 1806 of the sensor housing unit 1606 can be inserted into the bottom portion 1802 of the spacer 1684 or vice versa, as depicted by a vertical direction 1810. The spacer 1684 includes attachment mechanisms 1808 configured to engage with the protrusions 1806, e.g., to retain the sensor housing unit 1606 by the spacer 1684.

Different embodiments can include various containers including fluid containers or hydraulic cylinders. Different embodiments include various fluids including hydraulic fluids. Different embodiments can include different moveable objects (e.g., fluid interface elements) in the containers including a piston.

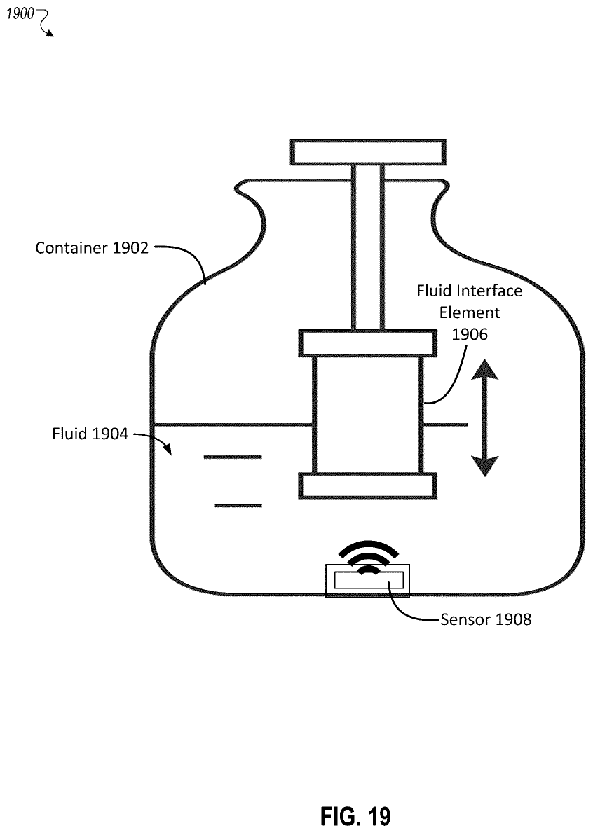

For example, FIG. 19 illustrates an example system 1900 for identifying features of a fluid 1904 in a container 1902. The system includes a container 1902 for a fluid 1904. The container 1902 can refer to variety of different vessels, receptacle, or enclosure capable of holding a fluid. This includes but is not limited to tanks, reservoirs, bladders, cylinders, bottles, pouches, or other structure suitable for containing liquids or gases. The fluid 1904 can refer to a variety of different liquid or semi-liquid substances, including those suitable for use in mechanical or automotive systems. Non-limiting examples of the fluid can include engine oil, coolant, transmission fluid, brake fluid, hydraulic fluid, and fuel. The fluid 1904 can vary in viscosity, composition, and function depending on the specific application. Similarly, the fluid interface element 1906 can include a variety of structures that move at least partially within the container 1902. The fluid interface element 1906 can be an element that slides, oscillates, or displaces the fluid 1904 in the container 1902 (e.g., such as a piston or plunger). The sensing unit 1908 can be the same or similar to the sensing unit 108, illustrated and described in reference to FIG. 1. In some embodiments, the sensing unit can include the sensor assembly block assembly 1502 illustrated and describe in reference to FIGS. 15-18B.

In some examples, the system 1900 operates with a computing device (not shown) in communication with the sensing unit 1908. The sensing unit 1908 can be configured to emit a radar signal through the fluid and collect a reflected signal off of the fluid interface element 1906. In some embodiments, the computing system is able to determine the location of the movable object 1906 based on the collected signals. Other features can also be identified. In some examples, the collected signals are compared to a previously collected signal. In some examples, a comparison between collected signals at different times can account for the determined position of the movable object at each of the times when the collected signals are detected by the sensing unit 1908.

In one example, this computing system can include a memory configured to store instructions, and one or more processors configured to execute the instructions to perform operations comprising for moving the fluid interface element 1906 to a defined position within the container 1902; emitting, by the sensing unit 1908, a radar signal through the fluid 1904 in the container 1902; collecting, at the sensing unit 1908, a reflected signal corresponding to the emitted radar signal, comparing the reflected signal to a previously collected signal, wherein the previously collected signal was collected by the sensing unit 1908 while the fluid interface clement 1906 was previously located at the defined position within the container 1902, and based on the comparing, identifying a feature of the fluid 1904.

In some embodiments, the identified feature can include an indication of a presence of a contaminant in the fluid 1904, such as water content, a presence of a residue, a presence of one or more particles. In some examples, identifying the presence of the one or more contaminants in the fluid 1904 can include detecting a difference in a time-of-flight associated with the reflected signal and a time-of-flight associated with the previously collected signal, wherein the detected difference is greater than a threshold difference. In some examples, identifying the presence of the one or more contaminants in the fluid 1904 can include detecting an attenuation in the reflected signal compared to the previously collected signal, wherein the attenuation is greater than a threshold attenuation level. In some examples, moving the fluid interface element 1908 to the defined position comprises moving the fluid interface element 1908 to a position of maximum extension. In some examples, identifying the presence of the one or more contaminants in the fluid 1904 can account for one or more properties of the fluid 1904 and/or a time when the fluid 1904 was last replaced. In some examples, the fluid 1904 within the container 1902 can be replaced subsequent to identifying the presence contaminants in the fluid 1904.

In some examples a signal (e.g., alert, notification, message) is generated to indicate that the presence of the one or more contaminants (or other feature) in the fluid 1904 was identified. In some examples, this signal provides an indication to a user to take an action with the container 1902 or fluid 1904. In one non-limiting example, the signal can indicate to a user that it is time to replace the fluid, or otherwise service system 1900. In another non-limiting example, the signal may trigger the system 1900 to automatically take an action (e.g., via the computing system).

Some of the examples described herein include or are defined by the following implementations.

Implementation A1 is a method for monitoring a hydraulic fluid comprising: moving a piston to a defined position within a hydraulic cylinder; emitting, by a radar sensing unit, a radar signal through the hydraulic fluid in the hydraulic cylinder; collecting, at the radar sensing unit, a reflected signal corresponding to the emitted radar signal; comparing the reflected signal to a previously collected signal, wherein the previously collected signal was collected by the radar sensing unit while the piston was previously located at the defined position within the hydraulic cylinder; and based on the comparing, identifying a presence of one or more contaminants in the hydraulic fluid.

Implementation A2 is the method of implementation A1, wherein the one or more contaminants comprise at least one of (i) water content in the hydraulic fluid, (ii) a presence of residues, or (iii) a presence of one or more particles in the hydraulic fluid.

Implementation A3 is the method of any of implementations A1-A2, wherein identifying the presence of the one or more contaminants in the hydraulic fluid comprises detecting a difference in a time-of-flight associated with the reflected signal and a time-of-flight associated with the previously collected signal, wherein the detected difference is greater than a threshold difference.

Implementation A4 is the method of any of implementations A1-A3, wherein identifying the presence of the one or more contaminants in the hydraulic fluid comprises detecting an attenuation in the reflected signal compared to the previously collected signal, wherein the attenuation is greater than a threshold attenuation level.

Implementation A5 is the method of any of implementations A1-A4, wherein moving the piston to the defined position within the hydraulic cylinder comprises moving the piston to a position of maximum extension.

Implementation A6 is the method of any of implementations A1-A5, wherein identifying the presence of the one or more contaminants in the hydraulic fluid comprises accounting for one or more properties of the hydraulic fluid and/or a time when the hydraulic fluid was last replaced.

Implementation A7 is the method of any of implementations A1-A6, further comprising replacing the hydraulic fluid within the hydraulic cylinder subsequent to identifying the presence of the one or more contaminants in the hydraulic fluid.

Implementation A8 is the method of any of implementations A1-A7, further comprising generating a signal indicating that the presence of the one or more contaminants in the hydraulic fluid was identified.

Implementation B1 is a system comprising: a hydraulic cylinder comprising a piston; a hydraulic fluid within the hydraulic cylinder; a radar sensing unit; and a computing device comprising: a memory configured to store instructions, and one or more processors configured to execute the instructions to perform operations comprising: moving the piston to a defined position within the hydraulic cylinder; emitting, by the radar sensing unit, a radar signal through the hydraulic fluid in the hydraulic cylinder; collecting, at the radar sensing unit, a reflected signal corresponding to the emitted radar signal; comparing the reflected signal to a previously collected signal, wherein the previously collected signal was collected by the radar sensing unit while the piston was previously located at the defined position within the hydraulic cylinder; and based on the comparing, identifying a presence of one or more contaminants in the hydraulic fluid.

Implementation B2 is the system of implementation B1, wherein the one or more contaminants comprise at least one of (i) water content in the hydraulic fluid, (ii) a presence of residues, or (iii) a presence of one or more particles in the hydraulic fluid.

Implementation B3 is the system of any of implementations B1-B2, wherein identifying the presence of the one or more contaminants in the hydraulic fluid comprises detecting a difference in a time-of-flight associated with the reflected signal and a time-of-flight associated with the previously collected signal, wherein the detected difference is greater than a threshold difference.

Implementation B4 is the system of any of implementations B1-B3, wherein identifying the presence of the one or more contaminants in the hydraulic fluid comprises detecting an attenuation in the reflected signal compared to the previously collected signal, wherein the attenuation is greater than a threshold attenuation level.

Implementation B5 is the system of any of implementations B1-B4, wherein moving the piston to the defined position within the hydraulic cylinder comprises moving the piston to a position of maximum extension.

Implementation B6 is the system of any of implementations B1-B5, wherein identifying the presence of the one or more contaminants in the hydraulic fluid comprises accounting for one or more properties of the hydraulic fluid and/or a time when the hydraulic fluid was last replaced.

Implementation B7 is the system of any of implementations B1-B6, wherein the operations further comprise replacing the hydraulic fluid within the hydraulic cylinder subsequent to identifying the presence of the one or more contaminants in the hydraulic fluid.

Implementation B8 is the system of any of implementations B1-B7, wherein the operations further comprise generating a signal indicating that the presence of the one or more contaminants in the hydraulic fluid was identified.

Implementation C1 is one or more machine-readable storage devices having encoded thereon computer readable instructions for causing one or more processing devices to perform operations comprising: moving a piston to a defined position within a hydraulic cylinder; emitting, by a radar sensing unit, a radar signal through a hydraulic fluid in the hydraulic cylinder; collecting, at the radar sensing unit, a reflected signal corresponding to the emitted radar signal; comparing the reflected signal to a previously collected signal, wherein the previously collected signal was collected by the radar sensing unit while the piston was previously located at the defined position within the hydraulic cylinder; and based on the comparing, identifying a presence of one or more contaminants in the hydraulic fluid.

Implementation C2 is the one or more machine-readable storage devices of implementation C1, wherein the one or more contaminants comprise at least one of (i) water content in the hydraulic fluid, (ii) a presence of residues, or (iii) a presence of one or more particles in the hydraulic fluid.

Implementation C3 is the one or more machine-readable storage devices of any one of implementations C1-C2, wherein identifying the presence of the one or more contaminants in the hydraulic fluid comprises detecting a difference in a time-of-flight associated with the reflected signal and a time-of-flight associated with the previously collected signal, wherein the detected difference is greater than a threshold difference.

Implementation C4 is the one or more machine-readable storage devices of any one of implementations C1-C3, wherein identifying the presence of the one or more contaminants in the hydraulic fluid comprises detecting an attenuation in the reflected signal compared to the previously collected signal, wherein the attenuation is greater than a threshold attenuation level.

Implementation C5 is the one or more machine-readable storage devices of any one of implementations C1-C4, wherein moving the piston to the defined position within the hydraulic cylinder comprises moving the piston to a position of maximum extension.

Implementation C6 is the one or more machine-readable storage devices of any one of implementations C1-C5, wherein identifying the presence of the one or more contaminants in the hydraulic fluid comprises accounting for one or more properties of the hydraulic fluid and/or a time when the hydraulic fluid was last replaced.

Implementation C7 is the one or more machine-readable storage devices of any one of implementations C1-C6, wherein the operations further comprise replacing the hydraulic fluid within the hydraulic cylinder subsequent to identifying the presence of the one or more contaminants in the hydraulic fluid.

Implementation C8 is the one or more machine-readable storage devices of any one of implementations C1-C7, wherein the operations further comprise further comprise generating a signal indicating that the presence of the one or more contaminants in the hydraulic fluid was identified.

Implementation D1 is a method for monitoring fluid comprising: moving a fluid interface element to a defined position within a container; emitting, by a radar sensing unit, a radar signal through the fluid in the container; collecting, at the radar sensing unit, a reflected signal corresponding to the emitted radar signal; comparing the reflected signal to a previously collected signal, wherein the previously collected signal was collected by the radar sensing unit while the fluid interface element was previously located at the defined position within the container; and based on the comparing, identifying a presence of one or more contaminants in the fluid.

Implementation D2 is the method of implementation D1, wherein the one or more contaminants comprise at least one of (i) water content in the fluid, (ii) a presence of residues, or (iii) a presence of one or more particles in the fluid.

Implementation D3 is the method of any one of implementations D1-D2, wherein identifying the presence of the one or more contaminants in the fluid comprises detecting a difference in a time-of-flight associated with the reflected signal and a time-of-flight associated with the previously collected signal, wherein the detected difference is greater than a threshold difference.

Implementation D4 is the method of any one of implementations D1-D3, wherein identifying the presence of the one or more contaminants in the fluid comprises detecting an attenuation in the reflected signal compared to the previously collected signal, wherein the attenuation is greater than a threshold attenuation level.

Implementation D5 is the method of any one of implementations D1-D4, wherein moving the fluid interface element to the defined position comprises moving the fluid interface element to a position of maximum extension.

Implementation D6 is the method of any one of implementations D1-D5, wherein identifying the presence of the one or more contaminants in the fluid comprises accounting for one or more properties of the fluid and/or a time when the fluid was last replaced.

Implementation D7 is the method of any one of implementations D1-D6, further comprising replacing the fluid within the container subsequent to identifying the presence of the one or more contaminants in the fluid.

Implementation D8 is the method of any one of implementations D1-D7, wherein moving the fluid interface element to the defined position comprises moving the fluid interface element to any defined position within the range of motion of the fluid interface element.

Implementation D9 is the method of any one of implementations D1-D8, further comprising generating a signal indicating that the presence of the one or more contaminants in the fluid was identified.

Implementation E1 is a system comprising: a container comprising a fluid interface element; a fluid within the container; a radar sensing unit; and a computing device comprising: a memory configured to store instructions, and one or more processors configured to execute the instructions to perform operations comprising: moving the fluid interface element to a defined position within the container; emitting, by the radar sensing unit, a radar signal through the fluid in the container; collecting, at the radar sensing unit, a reflected signal corresponding to the emitted radar signal; comparing the reflected signal to a previously collected signal, wherein the previously collected signal was collected by the radar sensing unit while the fluid interface element was previously located at the defined position within the container; and based on the comparing, identifying a presence of one or more contaminants in the fluid.

Implementation E2 is the system of implementation E1, wherein the one or more contaminants comprise at least one of (i) water content in the fluid, (ii) a presence of residues, or (iii) a presence of one or more particles in the fluid.

Implementation E3 is the system of any one of implementations E1-E2, wherein identifying the presence of the one or more contaminants in the fluid comprises detecting a difference in a time-of-flight associated with the reflected signal and a time-of-flight associated with the previously collected signal, wherein the detected difference is greater than a threshold difference.

Implementation E4 is the system of any one of implementations E1-E3, wherein identifying the presence of the one or more contaminants in the fluid comprises detecting an attenuation in the reflected signal compared to the previously collected signal, wherein the attenuation is greater than a threshold attenuation level.

Implementation E5 is the system of any one of implementations E1-E4, wherein moving the fluid interface element to the defined position comprises moving the fluid interface element to a position of maximum extension.

Implementation E6 is the system of any one of implementations E1-E5, wherein identifying the presence of the one or more contaminants in the fluid comprises accounting for one or more properties of the fluid and/or a time when the fluid was last replaced.

Implementation E7 is the system of any one of implementations E1-E6, wherein the operations further comprise replacing the fluid within the container subsequent to identifying the presence of the one or more contaminants in the fluid.

Implementation E8 is the system of any one of implementations E1-E7, wherein moving the fluid interface element to the defined position comprises moving the fluid interface element to any defined position within the range of motion of the fluid interface element.

Implementation E9 is the system of any one of implementations E1-E8, wherein the operations further comprise generating a signal indicating that the presence of the one or more contaminants in the fluid was identified.

Implementation F1 is one or more machine-readable storage devices having encoded thereon computer readable instructions for causing one or more processing devices to perform operations comprising: moving a fluid interface element to a defined position within a container; emitting, by a radar sensing unit, a radar signal through a fluid in the container; collecting, at the radar sensing unit, a reflected signal corresponding to the emitted radar signal; comparing the reflected signal to a previously collected signal, wherein the previously collected signal was collected by the radar sensing unit while the fluid interface element was previously located at the defined position within the container; and based on the comparing, identifying a presence of one or more contaminants in the fluid.

Implementation F2 is the one or more machine-readable storage devices of implementation F1, wherein the one or more contaminants comprise at least one of (i) water content in the fluid, (ii) a presence of residues, or (iii) a presence of one or more particles in the fluid.

Implementation F3 is the one or more machine-readable storage devices of any one of implementations F1-F2, wherein identifying the presence of the one or more contaminants in the fluid comprises detecting a difference in a time-of-flight associated with the reflected signal and a time-of-flight associated with the previously collected signal, wherein the detected difference is greater than a threshold difference.

Implementation F4 is the one or more machine-readable storage devices of any one of implementations F1-F3, wherein identifying the presence of the one or more contaminants in the fluid comprises detecting an attenuation in the reflected signal compared to the previously collected signal, wherein the attenuation is greater than a threshold attenuation level.

Implementation F5 is the one or more machine-readable storage devices of any one of implementations F1-F4, wherein moving the fluid interface element to the defined position within the container comprises moving the fluid interface element to a position of maximum extension.

Implementation F6 is the one or more machine-readable storage devices of any one of implementations F1-F5, wherein identifying the presence of the one or more contaminants in the fluid comprises accounting for one or more properties of the fluid and/or a time when the fluid was last replaced.

Implementation F7 is the one or more machine-readable storage devices of any one of implementations F1-F6, wherein the operations further comprise replacing the fluid within the container subsequent to identifying the presence of the one or more contaminants in the fluid.

Implementation F8 is the one or more machine-readable storage devices of any one of implementations F1-F7, wherein moving the fluid interface element to the defined position within the container comprises moving the fluid interface element to any defined position within the range of motion of the fluid interface element.

Implementation F9 is the one or more machine-readable storage devices of any one of implementations F1-F8,

Implementation G1 is A system comprising: a container comprising a fluid interface element; a fluid within the container; a radar sensing unit; and a computing device comprising: a memory configured to store instructions, and one or more processors configured to execute the instructions to perform operations comprising: moving the fluid interface element to a defined position within the container; emitting, by the radar sensing unit, a radar signal through the fluid in the container; collecting, at the radar sensing unit, a reflected signal corresponding to the emitted radar signal; comparing the reflected signal to a previously collected signal, wherein the previously collected signal was collected by the radar sensing unit while the fluid interface element was previously located at the defined position within the container; and based on the comparing, identifying a feature of the fluid.

Implementation G2 is the system of implementation G1, wherein the feature includes an indication of a presence of one or more contaminants in the fluid.

Implementation G3 is the system of implementation G2, wherein the one or more contaminants comprise at least one of (i) water content in the fluid, (ii) a presence of residues, or (iii) a presence of one or more particles in the fluid.

Implementation G4 is the system of any one of implementations G1-G3, wherein identifying the feature of the fluid comprises detecting a difference in a time-of-flight associated with the reflected signal and a time-of-flight associated with the previously collected signal, wherein the detected difference is greater than a threshold difference.

Implementation G5 is the system of any one of implementations G1-G4, wherein identifying the feature of the fluid comprises detecting an attenuation in the reflected signal compared to the previously collected signal, wherein the attenuation is greater than a threshold attenuation level.

Implementation G5 is the system of any one of implementations G1-G4, wherein moving the fluid interface element to the defined position comprises moving the fluid interface element to any defined position within the range of motion of the fluid interface element.

Implementation G6 is the system of any one of implementations G1-G5, wherein identifying the feature of the fluid comprises accounting for one or more properties of the fluid and/or a time when the fluid was last replaced.

Implementation G7 is the system of any one of implementations G1-G6, wherein the operations further comprise replacing the fluid within the container subsequent to identifying the feature of the fluid.

Implementation G8 is the system of any one of implementations G1-G7, wherein the operations further generating a signal indicating that the feature of the fluid was identified.

Other embodiments and applications not specifically described herein are also within the scope of the following claims. Elements of different implementations described herein may be combined to form other embodiments not specifically set forth above. Elements may be left out of the structures described herein without adversely affecting their operation. Furthermore, various separate elements may be combined into one or more individual elements to perform the functions described herein.

What is claimed is:

1. A method for monitoring a hydraulic fluid comprising:
moving a piston to a defined position within a hydraulic cylinder;
emitting, by a radar sensing unit, a radar signal through the hydraulic fluid in the hydraulic cylinder;
collecting, at the radar sensing unit, a reflected signal corresponding to the emitted radar signal;
comparing the reflected signal to a previously collected signal, wherein the previously collected signal was collected by the radar sensing unit while the piston was previously located at the defined position within the hydraulic cylinder; and
based on the comparing, identifying a presence of one or more contaminants in the hydraulic fluid.

2. The method of claim 1, wherein the one or more contaminants comprise at least one of (i) water content in the hydraulic fluid, (ii) a presence of residues, or (iii) a presence of one or more particles in the hydraulic fluid.

3. The method of claim 1, wherein identifying the presence of the one or more contaminants in the hydraulic fluid comprises detecting a difference in a time-of-flight associated with the reflected signal and a time-of-flight associated with the previously collected signal, wherein the detected difference is greater than a threshold difference.

4. The method of claim 1, wherein identifying the presence of the one or more contaminants in the hydraulic fluid comprises detecting an attenuation in the reflected signal compared to the previously collected signal, wherein the attenuation is greater than a threshold attenuation level.

5. The method of claim 1, wherein moving the piston to the defined position within the hydraulic cylinder comprises moving the piston to a position of maximum extension.

6. The method of claim 1, wherein identifying the presence of the one or more contaminants in the hydraulic fluid comprises accounting for one or more properties of the hydraulic fluid and/or a time when the hydraulic fluid was last replaced.

7. The method of claim 1, further comprising replacing the hydraulic fluid within the hydraulic cylinder subsequent to identifying the presence of the one or more contaminants in the hydraulic fluid.

8. The method of claim 1, further comprising generating a signal indicating that the presence of the one or more contaminants in the hydraulic fluid was identified.

9. The method of claim 1, wherein moving the piston to the defined position comprises moving the piston to any position within a range of motion of the piston.

10. A system comprising:
a hydraulic cylinder comprising a piston;
a hydraulic fluid within the hydraulic cylinder;
a radar sensing unit; and
a computing device comprising:
a memory configured to store instructions, and one or more processors configured to execute the instructions to perform operations comprising:
moving the piston to a defined position within the hydraulic cylinder;
emitting, by the radar sensing unit, a radar signal through the hydraulic fluid in the hydraulic cylinder;
collecting, at the radar sensing unit, a reflected signal corresponding to the emitted radar signal;
comparing the reflected signal to a previously collected signal, wherein the previously collected signal was collected by the radar sensing unit while the piston was previously located at the defined position within the hydraulic cylinder; and
based on the comparing, identifying a presence of one or more contaminants in the hydraulic fluid.

11. The system of claim 10, wherein the one or more contaminants comprise at least one of (i) water content in the hydraulic fluid, (ii) a presence of residues, or (iii) a presence of one or more particles in the hydraulic fluid.

12. The system of claim 10, wherein identifying the presence of the one or more contaminants in the hydraulic fluid comprises detecting a difference in a time-of-flight associated with the reflected signal and a time-of-flight associated with the previously collected signal, wherein the detected difference is greater than a threshold difference.

13. The system of claim 10, wherein identifying the presence of the one or more contaminants in the hydraulic fluid comprises detecting an attenuation in the reflected signal compared to the previously collected signal, wherein the attenuation is greater than a threshold attenuation level.

14. The system of claim 10, wherein moving the piston to the defined position within the hydraulic cylinder comprises moving the piston to a position of maximum extension.

15. The system of claim 10, wherein identifying the presence of the one or more contaminants in the hydraulic fluid comprises accounting for one or more properties of the hydraulic fluid and/or a time when the hydraulic fluid was last replaced.

16. The system of claim 10, wherein the operations further comprise generating a signal indicating that the presence of the one or more contaminants in the hydraulic fluid was identified.

17. One or more machine-readable storage devices having encoded thereon computer readable instructions for causing one or more processing devices to perform operations comprising:
moving a piston to a defined position within a hydraulic cylinder;
emitting, by a radar sensing unit, a radar signal through a hydraulic fluid in the hydraulic cylinder;
collecting, at the radar sensing unit, a reflected signal corresponding to the emitted radar signal;
comparing the reflected signal to a previously collected signal, wherein the previously collected signal was collected by the radar sensing unit while the piston was previously located at the defined position within the hydraulic cylinder; and
based on the comparing, identifying a presence of one or more contaminants in the hydraulic fluid.

18. The one or more machine-readable storage devices of claim 17, wherein the one or more contaminants comprise at least one of (i) water content in the hydraulic fluid, (ii) a presence of residues, or (iii) a presence of one or more particles in the hydraulic fluid.

19. The one or more machine-readable storage devices of claim 17, wherein identifying the presence of the one or more contaminants in the hydraulic fluid comprises detecting a difference in a time-of-flight associated with the reflected signal and a time-of-flight associated with the previously collected signal, wherein the detected difference is greater than a threshold difference.

20. The one or more machine-readable storage devices of claim 17, wherein identifying the presence of the one or more contaminants in the hydraulic fluid comprises detecting an attenuation in the reflected signal compared to the previously collected signal, wherein the attenuation is greater than a threshold attenuation level.
